(19) United States
(12) Patent Application Publication
(10) Pub. No.: US 2002/0115692 A1
(43) Pub. Date: Aug. 22, 2002

(54) Tryptophan derivatives

(76) Inventors: Sarah Catherine Archibald, Berkshire (GB); David Festus Charles Moffat, Berkshire (GB); Martin Clive Hutchings, Berkshire (GB); Anne Marie Foley, Berkshire (GB)

Correspondence Address: Woodcock Washburn LLP, 46th Floor, One Liberty Place, Philadelphia, PA, 19103, US

(21) Appl. No.: 09/989,840

(22) Filed: Nov. 20, 2001

(30) Foreign Application Priority Data
Nov. 21, 2000 (GB) 0028367.1

(51) Int. Cl. A61K 31/4439; A61K 31/405; C 07D 4 1/02; C07D 209/14

(52) U.S. Cl. 514/339; 514/415; 546/277.7; 548/503

(57) ABSTRACT

Tryptophan derivatives of formula (1) are described:

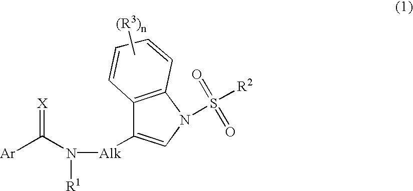

wherein:

Ar is an optionally substituted aromatic or heteroaromatic group;

X is an oxygen or sulphur atom;

Alk is a chain

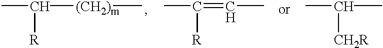

in which m is zero or the integer 1 or 2 and R is a carboxylic acid (—CO₂H) or a derivative or biostere thereof;

R² is an optionally substituted aliphatic group;

R³ is an optional substituent;

n is zero or the integer 1, 2 or 3;

and the salts, solvates, hydrates and N-oxides thereof.

The compounds are able to inhibit the binding of LFA-1 to its ligands and are of use in the prophylaxis and treatment of inflammatory diseases or disorders or autoimmune diseases.

This invention relates to a series of tryptophan derivatives and related compounds, to compositions containing them, to processes for their preparation and to their use in medicine.

A co-ordinated series of events beginning with vasodilation and increased vascular permeability together with exudation of fluid and plasma proteins results in inflammation. Simultaneously inflammatory cells infiltrate the site of inflammation, in response to inflammatory mediators generated at the site of initial lesion. Mediators that have chemotactic activity for leukocytes, the principle group of inflammatory cells, include chemokines such as IL-8, MCP-1, MIP-1 and RANTES, complement fragments and lipid mediators. By a process known as cell adhesion these circulating leukocytes localize at a precise point on the vascular endothelium, prior to crossing the endothelium to the site of inflammation.

Leukocytes are an important component of human peripheral blood that may be further divided into a number of different members. The leukocyte family consists of neutrophils, lymphocytes (B- and T-cell subtypes), monocytes, eosinophils and basophils. During an inflammatory response peripheral blood leukocytes are recruited to the site of inflammation or injury by a series of specific cellular interactions. A critical step in the inflammatory response is the adhesion of leukocytes to the vascular endothelium and migration from the circulation to the site of inflammation. The lymphocyte function associated antigen-1 (LFA-1, (α_{L}β₂, CD11a/CD18), which is present on the surface of all mature leukocytes except a subset of macrophages, has been identified as the major integrin that mediates lymphocyte adhesion and activation leading to a normal immune response as well as several pathologies (Springer, T. A., Nature, 1990, 346, 425-434). This cellular adhesion molecule belongs to the leukocyte-specific β₂ subfamily of integrins, in which a common β₂ subunit (CD18) is associated with distinct but homologous α subunits. Other members of the family include Mac-1 (α_{M}β₂, CD11b/CD18), p150.95 (α_{Xβ}₂, CD11c/CD18) and α_{D}β₂ (CD11d/CD18).

Amongst its many roles LFA-1 plays a key part in the adhesion to, and migration across vascular endotheiium of leukocytes, particularly monocytes and neutrophils. The first step consists of leukocytes rolling along the vascular endothelium in the region of inflammation, mediated by sialyl Lewis^{X} on leukocytes interacting with E- and P-selectins expressed on the endothelium. This reversible interaction is followed by the stronger interactions of the second stage which are mediated by the interaction of leukocyte integrins (LFA-1 and Mac-1) with intracellular adhesion molecules (predominantly ICAM-1) which are induced on the endothelium in response to inflammatory mediators such as IL-1, LPS and TNF-α. A conformational change in LFA-1 and Mac-1 in response to chemokines such as IL-8, MIP-1-α, RANTES and lipid mediators results in a firm attachment of leukocytes to endothelium. A third stage in which LFA-1 and Mac-1 also play a role is the extravasation of the leukocytes between the cells forming the blood vessel walls which is followed by a final stage of leukocyte migration along concentration gradients of chemokines secreted by cells at the site of infection or inflammation. A further role for LFA-1 is a part (in combination with the major histocompatability protein, MHC on antigen presenting cells) in the interaction of T-cell receptor with antigen on antigen-presenting cells. On binding to an antigen presented on a MHC signalling through the T-cell receptor induces a conformational change in LFA-1 which greatly increases its affinity for its ligands (ICAM-1 and ICAM-2) on the antigen-presenting cell so stabilizing the interaction between antigen-specific T-cell and antigen-presenting cell.

The ligands for LFA-1 were identified by functional studies. These ligands (intercellular adhesion molecules) are known as ICAM-1 (Rothlein, R. et al, J. Immunol., 1986, 137, 1270-1274; Staunton, D. E. et al, Cell, 1988, 52, 925-933), ICAM-2 (Staunton, D. E. et al, Nature, 1989, 339, 61-64), ICAM-3 (Fawcett, J. et al, Nature, 1992, 360, 481-484; Vazeux, R. et al, Nature, 1992, 360, 485-488; De Fougerolles, A. R. and Springer, T. A., J Exp. Med., 1990, 175, 185-190), ICAM-4 (Bailly, P. et al.,Eur. J. Immunol., 1995, 25, 3316-3320) and ICAM-5 (Tian, L. et al, J. Immunol., 1997, 158, 928-936). ICAM-1, -2, -3, -4 and -5 are members of the immunoglobulin (Ig) superfamily and contain five, two and five Ig-like domains respectively of which the first domains are necessary and sufficient for interaction with LFA-1 (Binnerts, M. E. and van Kooyk, Y. Immunology Today, 1999, 20, 240-245). These ICAMs have distinct tissue distributions (Binnerts, M. E. et al, Eur. J. Immunol., 1994, 24, 2155-2160). For instance ICAM-1 is expressed on a variety of hematopoietic and non-hematopoietic cells including activated leukocytes, dermal fibroblasts and vascular endothelial cells (Dustin, M. L. et al, J. Immunol. 1986, 137, 245-254) and is upregulated at sites of inflammation by a variety of inflammatory mediators including LPS, IL-1 and TNF (Dustin, M. L. et al, J. Cell Biol. 1988, 107, 321-331).

Binding of LFA-1 to ICAMs mediates a range of lymphocyte functions including T-lymphocyte mediated target cell lysis, lymphokine production of helper T-cells in response to antigen presenting cells, immunoglobulin production through T-cell-B-cell interactions and natural killing of tumour cells. More specifically the LFA-1/ICAM-1 interaction is known to play a part in lymphocyte adhesion (Dustin, M. L. et al, J. Cell Biol. 1988, 107, 321-331), monocyte adhesion (Arnaout, M. A. et al, J. Cell Physiol. 1988, 137, 305-309) and polymorphonuclear leukocyte adhesion (Lo, S. K. et al, J. Immunol., 1989, 143, 3325-3329) to endothelial cells. Interactions of LFA-1 with ICAM-2 are thought to mediate natural killer cell activity (Helander, T. S. et al Nature, 1996, 382, 265-268) and interaction with ICAM-3 is believed to have a role in the initiation of the immune response (Simmons, D. L., Cancer Surveys, Cell Adhesion and Cancer, 1995, 24, 141-155).

Furthermore functional studies using monoclonal antibodies have demonstrated that members of the CD11/CD18 integrin family mediate a variety of cell-cell interactions including those that occur during inflammation (Springer, T. A. et al, Ann. Rev. Immunol., 1987, 5, 223-252). Thus monoclonal antibodies to LFA-1 have been shown to inhibit T-cell activation (Dougherty, G. J. and Hogg, N. Eur. J. Immunol., 1987, 17, 943-47; Kuypers et al, Res. Immunol., 1989, 140, 461), conjugate formation required for antigen-specific cytotoxic T-lymphocyte mediated killing (Kishimoto T. K. et al, Adv. Immunol., 1989, 46, 149-182), natural killer (NK) cell killing (Krensky, A. M., et al, J. Immunol., 1983, 131, 611-616), the mixed lymphocyte response and T-cell dependent B-cell proliferation and differentiation (Davignon, D. et al, J. Immunol., 1981, 127, 590-595). Additionally such antibodies have been demonstrated to block T lymphoblast (Dustin, M. L. et al, J. Cell Biol., 1988, 107, 321-331) and neutrophil adhesion to endothelial cells (Smith, C. W. et al, J. Clin. Invest., 1989, 83, 2008-2017) including adhesion of T-cells to vascular endothelium (Hogg, N. and Landis, R. C., Curr. Opin. Immunol., 1993, 5, 383-390 and Picker, L. J. , Curr. Opin. Immunol., 1994, 6, 394-406). Antibodies blocking CD18 or ICAM-1 activity are the subject of for example International Patent Specifications Nos. WO093/02191, WO094/02175, WO094/12214, WO097/26912 and U.S. Pat. No. 5,695,760.

Following on from these observations that the LFA-1:ICAM-1 interaction is necessary for optimal T-cell function and the finding that anti-CD11a monoclonal antibodies (Mabs) can prolong graft survival in mice (Heagly et al, Transplantation, 1984, 37, 520-523) Mabs to CD11a have been tested for prevention of graft rejection in primates and humans. Thus in vivo administration of an anti-CD11a Mab prolonged skin allograft survival in cynomologous monkeys (Berlin, P. J. et al, Transplantation, 1992, 53, 840-849) and rat anti-murine CD11a antibody was effective in controlling steroid-resistant graft-versus-host disease in humans (Stoppa et al, Transplant. Int., 1991, 4, 3-7). Mabs are also efficacious in models of skin inflammation, e.g. models of contact hypersensitivity in mice (Scheynius, A. et al, J. Immunol, 1996, 156, 1804-1809). In humans, efficacy has been observed with anti-LFA-1 Mab in patients with psoriasis (Gottlieb, A. et al, J. Am. Acad. Dermatol., 2000, 42, 428-435).

It has further been demonstrated that antisense oligonucleotides to murine ICAM-1 can attenuate reperfusion injury and renal failure in rats (Stepkowski, S. M. et al, J. Immunol, 1994, 153, 5336-46; Haller et al, Kidney Int., 1996, 50, 473-480) and molecules of this type have been patented (for example U.S. Pat. Nos. 5,591,623 and 5,580,969).

The use of biological molecules such as antibodies and oligonucleotides in treating inflammatory disease mediated by LFA-1 is not ideal since molecules of this type can suffer from lack of stability, low bioavailability, immunogenecity problems, high cost and possible risk of serious side effects. It is therefore preferable to use low molecular weight antagonists of the interaction between LFA-1 and its ligands since these molecules do not suffer from the same disadvantages as biological molecules. Hence such small molecules that block LFA-1 activity and so alter leukocyte trafficking are desirable as therapeutic agents for the treatment of chronic and acute inflammatory diseases or disorders and autoimmune diseases.

We have now found such a group of compounds which are potent inhibitors of the interaction between LFA-1 and ICAM-1. Such compounds are of use in medicine, for example in the prophylaxis and treatment of disorders involving inappropriate leukocyte trafficking and in acute and chronic inflammatory disorders as described herein.

Thus according to one aspect of the invention we provide a compound of formula (1)

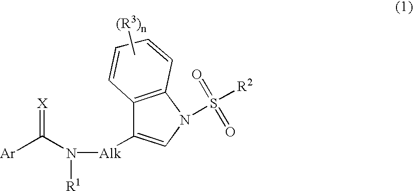

wherein:

Ar is an optionally substituted aromatic or heteroaromatic group;

X is an oxygen or sulphur atom;

Alk is a chain

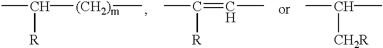

in which m is zero or the integer 1 or 2 and R is a carboxylic acid (—CO₂H) or a derivative or biostere thereof;

R¹ is a hydrogen atom or a C₁₋₆alkyl group;

R² is an optionally substituted aliphatic group;

R³ is an atom or group —L¹(Alk¹)ₜL²(R⁴)_{U} in which L¹ and L² which may be the same or different is each a covalent bond or a linker atom or group, t is zero or the integer 1, u is an integer 1, 2 or 3, Alk¹ is an aliphatic or heteroaliphatic chain and R⁴ is a hydrogen or halogen atom or a group selected from alkyl, —OR⁵ [where R⁵ is a hydrogen atom or an optionally substituted alkyl group], —SR⁵, —NR⁵R⁶[where R⁶ is as just defined for R⁵ and may be the same or different], —NO₂, —CN, —CO₂R⁵, —SO₃H, —SOR⁵, —SO₂R⁵, —SO₃R⁵, —OCO₂R⁵, —CONR⁵R⁶, —OCONR⁵R⁶, —CSNR⁵R⁶, —COR⁵, —OCOR⁵, —N(R⁵)COR⁶, N(R⁵)CSR⁶, —N(R⁵)CO₂R⁶, —SO₂N(R⁵)(R⁶), —N(R⁵)SO₂R⁶, —N(R⁵)CON(R⁶)(R⁷) [where R⁷ is a hydrogen atom or an optionally substituted alkyl group], —N(R⁵)CSN(R⁶)(R⁷) or —N(R⁵)SO₂N(R⁶)(R⁷), provided that when t is zero and each of L¹ and L² is a covalent bond then u is the integer 1 and R⁴ is other than a hydrogen atom;

n is zero or the integer 1, 2 or 3;

and the salts, solvates, hydrates and N-oxides thereof.

It will be appreciated that certain compounds of formula (1) may exist as geometric isomers (E or Z isomers) The compounds may also have one or more chiral centres, and exist as enantiomers or diastereomers. The invention is to be understood to extend to all such geometric isomers, enantiomers, diastereomers and mixtures thereof, including racemates. Formula (1) and the formulae hereinafter are intended to represent all individual isomers and mixtures thereof, unless stated or shown otherwise. In addition, compounds of formula (1) may exist as tautomers, for example keto (CH₂C=O)—enol (CH=CHOH) tautomers. Formula (1) and the formulae hereinafter are intended to represent all individual tautomers and mixtures thereof, unless stated otherwise.

The compounds of formula (1) are potent and selective inhibitors of β2 integrins such as LFA-1. Members of the group are able to inhibit the action of LFA-1 at concentrations at which they generally have no or minimal action on, β integrins of other subgroups. The compounds are thus of use in medicine, for example in the prophylaxis and treatment of immune and inflammatory disorders as described hereinafter.

In the compounds of the invention as represented by formula (1) and the more detailed description hereinafter certain of the general terms used in relation to substituents are to be understood to include the following atoms or groups unless specified otherwise.

Thus as used herein the term “alkyl”, whether present as a group or part of a group includes straight or branched C₁₋₁₀alkyl groups, for example C₁₋₆alkyl groups such as methyl, ethyl, n-propyl, i-propyl, n-butyl, i-butyl, s-butyl or t-butyl groups and C₃₋₁₀cycloalkyl groups, for example C₃₋₇cycloalkyl groups such as cyclopropyl, cyclobutyl, cyclopentyl and cyclohexyl groups. Similarly, the terms “alkenyl” or “alkynyl” are intended to mean straight or branched C₂₋₁₀alkenyl, C₃₋₁₀cycloalkenyl or C₂₋₁₀alkynyl groups such as C₂₋₆alkenyl, C₃₋₇cycloalkenyl or C₂₋₆alkynyl groups, for example —CHCH₂, —CHCHCH₃, —CH₂CHCHCH₃, —CCH, —CH₂CCH, —CH₂CCCH₃, cyclopentenyl or cyclohexenyl groups. Each of these groups may be optionally substituted on any carbon atom. Optional substituents that may be present include those optional substituents mentioned hereinafter in relation to optionally substituted aliphatic groups.

The term “halogen atom” is intended to include fluorine, chlorine, bromine or iodine atoms.

The term “haloalkyl” is intended to include the alkyl groups just mentioned substituted by one, two or three of the halogen atoms just described. Particular examples of such groups include —CF₃, —CCl₃, —CHF₂, —CHCl₂, —CH₂F, and —CH₂Cl groups.

The term “alkoxy” as used herein is intended to include straight or branched C₁₋₁₀alkoxy for example C₁₋₆alkoxy such as methoxy, ethoxy, n-propoxy, i-propoxy and t-butoxy. “Haloalkoxy” as used herein includes any of those alkoxy groups substituted by one, two or three halogen atoms as described above. Particular examples include —OCF₃, —OCCl₃, —OCHF₂, —OCHCl₂, —OCH₂F and —OCH₂Cl groups.

As used herein the term “alkylthio” is intended to include straight or branched C₁₋₁₀alkylthio, e.g. C₁₋₆alkylthio such as methylthio or ethylthio groups.

The term “aliphatic group” is intended to include optionally substituted straight or branched C₁₋₁₀alkyl, e.g. C₁₋₆alkyl, C₂₋₁₀alkenyl e.g. C₂₋₆alkenyl or C₂₋₁₀alkynyl e.g. C₂₋₆alkynyl groups.

The term “heteroaliphatic group” is intended to include the optionally substituted aliphatic groups just described but with each group additionally containing one, two, three or four heteroatoms or heteroatom-containing groups. Particular heteroatoms or groups include atoms or groups L³ where L³ is a linker atom or group. Each L³ atom or group may interrupt the aliphatic group, or may be positioned at its terminal carbon atom to connect the group to an adjoining atom or group. Particular examples of suitable L³ atoms or groups include —O— or —S— atoms or —C(O)—, —C(O)O—, —C(S)—, —S(O), —S(O)₂—, —N(R⁸)— [where R⁸ is a hydrogen atom or an alkyl group], —N(R⁸)N(R⁸)—, —N(R⁸)O—, —CON(R⁸)—, —OC(O)N(R⁸)—, —CSN(R⁸)—, —N(R⁸)CO—, —N(R⁸)C(O)O—, —N(R⁸)CS—, —S(O)₂N(R⁸)—, —N(R⁸)S(O)₂—, —N(R⁸)CON(R⁸)—, —N(R⁸)CSN(R⁸)—, or —N(R⁸)SO₂N(R⁸)— groups. Where the linker group contains two R⁸ substituents, these may be the same or different.

Particular examples of aliphatic groups include optionally substituted —CH₃, —CH₂CH₃, —CH(CH₃)₂, —(CH₂)₂CH₃, —(CH₂)₃CH₃, —CH(CH₃)CH₂CH₃, —CH₂CH(CH₃)₂, —C(CH₃)₃, —(CH₂)₄CH₃, —(CH₂)₅CH₃, —CHCH₂, —CHCHCH₃, —CH₂CHCH₂, —CHCHCH₂CH₃, —CH₂CHCHCH₃, —(CH₂)₂CHCH₂, —CCH, —CCCH₃, —CH₂CCH, —CCCH₂CH₃, —CH₂CCCH₃, or —(CH₂)₂CCH groups. Where appropriate each of said groups may be optionally interrupted by one, two, three or more atoms and/or groups L³ to form an optionally substituted heteroaliphatic group. Particular examples include optionally substituted —L³CH₃, —CH₂L³CH₃, —L³CH₂CH₃, —L³CH₂CHCH₂, —L³CH₂CCH, —CH₂L³CH₂CH₃, —L³CH₂L³CH₃, —(CH₂)₂L³CH₃, —L³(CH₂)₂CH₃ and —(CH₂)₂L³CH₂CH₃ groups.

The optional substituents which may be present on aliphatic or heteroaliphatic groups include one, two, three or more substituents where each substituent may be the same or different and is selected from halogen atoms, or alkoxy, haloalkoxy, hydroxy (—OH), thiol (—SH), alkylthio, amino (—NH₂), substituted amino, —CN, —CO₂H, —CO₂R⁹ (where R⁹ is an optionally substituted alkyl group), —SO₃H, —SOR⁹, —SO₂R⁹, —SO₃R⁹, —OCO₂R⁹, —C(O)H, —C(O)R⁹, —OC(O)R⁹, —C(S)R⁹, —C(O)N(R¹⁰)(R¹⁰)(R¹¹) (where R¹⁰ and R¹¹, which may be the same or different is each a hydrogen atom or an optionally substituted alkyl group), —OC(O)N(R¹⁰)(R¹¹), —N(R¹⁰)C(O)R¹¹, —CSN(R¹⁰)(R¹¹), —N(R¹⁰)C(S)(R¹¹), —SO₂N(R¹⁰)(R¹¹), —N(R¹⁰)SO₂R¹¹, —N(R¹⁰)C(O)N(R¹¹)(R¹²) (where R¹² is a hydrogen atom or an alkyl group), —N(R¹⁰)C(S)N(R¹¹)(R¹²) or —N(R¹⁰)SO₂N(R¹¹)(R¹²). Substituted amino groups include —NHR⁹ and —N(R⁹)(R¹⁰) groups.

It will be understood that the terms optionally substituted aliphatic or heteroaliphatic chain include those optionally substituted aliphatic and heteroaliphatic groups just described where a terminal hydrogen atom is replaced by a covalent bond. Thus for example alkyl, alkenyl and alkynyl chains become alkylenyl, alkenylenyl and alkynylenyl chains respectively. Particular non-limiting examples include a —CH₂CH₃ group becoming a —CH₂CH₂— chain and a —L³(CH₂)₂CH₃ group becoming a —L³(CH₂)₃— chain.

The term “aromatic group” is intended to include for example optionally substituted monocyclic or bicyclic fused ring C₆₋₁₂aromatic groups, such as phenyl, 1- or 2-naphthyl, 1- or 2-tetrahydronaphthyl, indanyl or indenyl groups. Each of these aromatic groups may be optionally substituted by one, two, three or more R¹³ atoms or groups as defined below.

The term “heteroaromatic group” is intended to include for example optionally substituted C₁₋₉heteroaromatic groups containing for example one, two, three or four heteroatoms selected from oxygen, sulphur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulphur or nitrogen atoms. Bicyclic heteroaromatic groups include for example eight- to thirteen-membered fused-ring heteroaromatic groups containing one, two or more heteroatoms selected from oxygen, sulphur or nitrogen atoms.

Particular examples of heteroaromatic groups of these types include pyrrolyl, furyl, thienyl, imidazolyl, N-C₁₋₆alkylimidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-thiadiazole, pyridyl, pyrimidinyl, pyridazinyl, pyrazinyl, 1,3,5-triazinyl, 1,2,4-triazinyl, 1,2,3-triazinyl, benzofuryl, [2,3-dihydro]benzofuryl, benzothienyl, benzotriazolyl, indolyl, indolinyl, isoindolyl, indazolinyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, qunoxalinyl, naphthyridinyl, pyrido[3,4-b]pyridyl, pyrido[3,2-b]pyridyl, pyrido[4,3-b]-pyridyl, quinolinyl, isoquinolinyl, phthalazinyl, tetrazolyl, 5,6,7,8-tetrahydroquinolinyl, 5,6,7,8-tetrahydroisoquinolinyl, and imidyl, e.g. succinimidyl, phthalimidyl, or naphthalimidyl such as 1,8-naphthalimidyl. Each of these heteroaromatic groups may be optionally substituted by one, two, three or more R¹³ atoms or groups as defined below.

The aromatic and heteroaromatic groups may be attached to the remainder of the compound of formula (1) by any carbon or hetero e.g. nitrogen atom as appropriate.

When Ar is an optionally substituted pyridyl group it may include any pyridyl group of formula (1a):

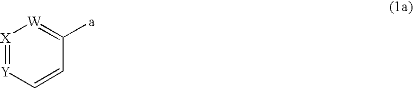

where a represents the point of attachment to the rest of the molecule of formula (1), one of W, X and Y is a N atom and the other two are CH groups. When Ar is an optionally substituted phenyl group it may for example have the formula (1a) in which each of W, X and Y is a CH group. The hydrogen atom of any of the CH groups present in pyridyl or phenyl groups of formula (1a) may be optionally replaced by any R¹³ atom or group as described below.

Optional substituents which may be present on any carbon atom of the aromatic or heteroaromatic groups represented by Ar in compounds of formula (1) include one, two, three or more substituents, each selected from an atom or group R¹³ in which R¹³ is —R^{13a}or —Alk²(R^{13a})_{f}, where R ^{13a} is a halogen atom, or an amino (—NH₂), substituted amino, nitro, cyano, amidino, hydroxyl (—OH), substituted hydroxyl, formyl, carboxyl (—CO₂H), esterified carboxyl, thiol (—SH), substituted thiol, —COR¹⁴ [where R¹⁴ is an —Alk²(R^{13a})_{f} group], —CSR¹⁴, —SO₃H, —SOR¹⁴, —SO₂R¹⁴, —SO₃R¹⁴, —SO₂NH₂, —SO₂NHR¹⁴, —SO₂N(R¹⁴)₂, —CONH₂, —CSNH₂, —CONHR¹⁴, —CSNHR¹⁴, —CON(R¹⁴)₂, —CSN(R¹⁴)₂, —N(R¹⁵)SO₂R¹⁴, [where R¹⁵ is a hydrogen atom or an optionally substituted alkyl group] —N(SO₂R¹⁴)₂, —N(R¹⁵)SO₂NH₂, —N(R¹⁵)SO₂NHR¹⁴, —N(R¹⁵)SO₂N(R¹⁴)₂, —N(R¹⁵)COR¹⁴, —N(R¹⁵)CONH₂, —N(R¹⁵)CONHR¹⁴, —N(R¹⁵)CON(R¹⁴)₂, —N(R¹⁵)CSNH₂, —N(R¹⁵)CSNHR¹⁴, —N(R¹⁵)CSN(R¹⁴)₂, —N(R¹⁵)CSR¹⁴, —N(R¹⁵)C(O)OR¹⁴ or optionally substituted C₆₋₁₂aromatic or C₁₋₉heteroaromatic group; Alk² is a straight or branched C₁₋₆alkylene, C₂₋₆alkenylene or C₂₋₆alkynylene chain, optionally interrupted by one, two or three —O— or —S— atoms or —S(O)g— [where g is an integer 1 or 2] or —N(R¹⁵)— groups; and f is zero or an integer 1, 2 or 3. It will be appreciated that when two R¹⁴ or R¹⁵ groups are present in one of the above substituents, the R¹⁴ or R¹⁵ groups may be the same or different.

When in the group —Alk²(R^{13a})_{f} f is an integer 1, 2 or 3, it is to be understood that the substituent or substituents R^{13a} may be present on any suitable carbon atom in —Alk². Where more than one R^{13a} substituent is present these may be the same or different and may be present on the same or different atom in —Alk². Clearly, when f is zero and no substituent R^{13a} is present the alkylene, alkenylene or alkynylene chain represented by Alk² then Alk² becomes an alkyl, alkenyl or alkynyl group.

When R^{13a} is a substituted amino group it may be for example a group —NHR¹⁴ [where R¹⁴ is as defined above] or a group —N(R¹⁴)₂ wherein each R¹⁴ group is the same or different.

When R^{13a} is a substituted hydroxyl or substituted thiol group it may be for example a group —OR¹⁴ or a —SR¹⁴ or —SC(=NH)NH₂ group respectively.

When R^{13a} is an optionally substituted C₆₋₁₂aromatic group it may be for example an optionally substituted phenyl group. When R^{13a} is an optionally substituted C₁₋₉heteroaromatic group it may be for example an optionally substituted furanyl, thienyl, pyrrolyl, thiazolyl, imidazolyl, pyrazolyl, triazolyl, pyridyl or pyrimidinyl group. Optional substituents that may be present on such aromatic or heteroaromatic groups include those R¹³ atoms and groups as just described.

Esterified carboxyl groups represented by the group R^{13a} include groups of formula —CO₂Alk⁵ wherein Alk⁵ is a straight or branched optionally substituted C₁₋₈alkyl group such as a methyl, ethyl, n-propyl, i-propyl, n-butyl, i-butyl, s-butyl or t-butyl group; an optionally substituted C₂₋₈alkenyl group such as a propenyl e.g. 2-propenyl or butenyl e.g. 2-butenyl or 3-butenyl group, an optionally substituted C₂₋₈alkynyl group such as a ethynyl, propynyl e.g. 2-propynyl or butynyl e.g. 2-butynyl or 3-butynyl group, an optionally substituted C₃₋₈cycloalkyl group such as a cyclopropyl, cyclobutyl, cyclopentyl, cyclohexyl or cycloheptyl group; an optionally substituted C₃₋₈cycloalkylC₁₋₈alkyl group such as a cyclopentylmethyl, cyclohexylmethyl or cyclohexylethyl group; an optionally substituted C₃₋₈heterocycloalkylC₁₋₆alkyl group such as a morpholinyl-N-ethyl, thiomorpholinyl-N-methyl, pyrrolidinyl-N-ethyl, pyrrolidinyl-N-propyl, piperidinyl-N-ethyl, pyrazolidinyl-N-methyl or piperazinyl-N-ethyl group; an optionally substituted C₁₋₆alkyloxyC₁₋₆alkyl group such as a methyloxyethyl or propyloxyethyl group; an optionally substituted C₁₋₆alkylthioC₁₋₆alkyl group such as an ethylthioethyl group; an optionally substituted C₁₋₆alkylsulfinylC₁₋₆alkyl group such as an methylsulfinylethyl group; an optionally substituted C₁₋₆alkylsulfonylC₁₋₆alkyl group such as an methylsulfonylmethyl group; an optionally substituted C₃₋₈cycloalkyloxyC₁₋₆alkyl group such as a cyclohexyloxymethyl group; an optionally substituted C₃₋₈cycloalkylthioC₁₋₆alkyl group such as a cyclopentylthiomethyl group; an optionally substituted C₃₋₈cycloalkylsulfinylC₁₋₆alkyl group such as a cyclopentylsulfinylmethyl group; an optionally substituted C₃₋₈cycloalkylsulfonylC₁₋₆alkyl group such as a cyclopentylsulfonylmethyl group; an optionally substituted C₁₋₆alkyloxycarbonylC₁₋₆alkyl group such as isobutoxycarbonylpropyl group; an optionally substituted C₁₋₆alkyloxycarbonylC₁₋₆alkenyl group such as isobutoxycarbonylpentenyl group; an optionally substituted C₁₋₆alkyloxycarbonyloxyC₁₋₆alkyl group such as an isopropoxycarbonyloxyethyl e.g a 1-(isopropoxycarbonyloxy)ethyl, 2-(isopropoxycarbonyloxy)ethyl or ethyloxycarbonyloxymethyl group; an optionally substituted C₁₋₆alkyloxycarbonyloxyC,₁₋₆alkenyl group such as a isopropoxycarbonyloxybutenyl group, an optionally substituted C₃₋₈cycloalkyloxycarbonyloxyC₁₋₆alkyl group such as a cyclohexyloxycarbonyloxyethyl, e.g. a 2-(cyclohexyloxycarbonyloxy)ethyl group, an optionally substituted N-di-C₁₋₈alkylaminoC₁₋₈alkyl group such as a N-dimethylaminoethyl or N-diethylaminoethyl group; an optionally substituted N-C₆₋₁₂aryl-N-C₁₋₆alkylaminoC₁₋₆alkyl group such as a N-phenyl-N-methylaminomethyl group; an optionally substituted N-di-C₁₋₈alkylcarbamoylC₁₋₈alkyl group such as a N-diethylcarbamoylmethyl group; an optionally substituted C₆₋₁₀arylC₁₋₆alkyl group such as an optionally substituted benzyl, phenylethyl, phenylpropyl, 1-naphthylmethyl or 2-naphthylmethyl group; a C₆₋₁₀aryl group such as an optionally substituted phenyl, 1-naphthyl or 2-naphthyl group; a C₆₋₁₀aryloxyC₁₋₈alkyl group such as an optionally substituted phenyloxymethyl, phenyloxyethyl, 1-naphthyloxymethyl, or 2-naphthyloxymethyl group; a C₆₋₁₂arylthioC₁₋₈alkyl group such as an optionally substituted phenylthioethyl group; a C₆₋₁₂arylsulfinylC₁₋₈alkyl group such as an optionally substituted phenylsulfinylmethyl group; a C₆₋₁₂arylsulfonylC₁₋₈alkyl group such as an optionally substituted phenylsulfonylmethyl group; an optionally substituted C₁₋₈alkanoyloxyC₁₋₈alkyl group, such as a acetoxymethyl, ethoxycarbonyloxyethyl, pivaloyloxymethyl, propionyloxyethyl or propionyloxypropyl group; an optionally substituted C₄₋₈imidoC, ₁₋₈alkyl group such as a succinimidomethyl or phthalamidoethyl group; a C₆₋₁₂aroyloxyC_{1 -8}alkyl group such as an optionally substituted benzoyloxyethyl or benzoyloxypropyl group or a triglyceride such as a 2-substituted triglyceride e.g. a 1,3-di-C₁₋₈alkylglycerol-2-yl group such as a 1,3-diheptylglycerol-2-yl group. Optional substituents present on the Alk⁵ group include R^{13a} substituents described above.

It will be appreciated that in the forgoing list of Alk⁵ groups the point of attachment to the remainder of the compound of formula (1) is via the last described part of the Alk⁵ group. Thus, for example a methoxyethyl group would be attached by the ethyl group, whilst a morpholinyl-N-ethyl group would be attached via the N-ethyl group.

It will be further appreciated that in the forgoing list of Alk⁵ groups, where not specifically mentioned, alkyl groups may be replaced by alkenyl or alkynyl groups where such groups are as previously defined. Additionally these alkyl, alkenyl or alkynyl groups may optionally be interrupted by one, two or three linker atoms or groups where such linker atoms and groups are as previously defined for L³.

When Alk² is present in or as a substituent it may be for example a methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene or 3-butynylene chain, optionally interrupted by one, two, or three —O— or —S— , atoms or —S(O)—, —S(O)₂— or —N(R¹⁵)— groups.

Particularly useful atoms or groups represented by R¹³ include fluorine, chlorine, bromine or iodine atoms, or C₁₋₆alkyl, e.g. methyl, ethyl, n-propyl, i-propyl, n-butyl or t-butyl, C₁₋₆hydroxyalkyl, e.g. hydroxymethyl or hydroxyethyl, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, haloC₁₋₆alkyl, e.g. trifluoromethyl, haloC₁₋₆alkoxy, e.g. trifluoromethoxy, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, aminoC₁₋₆alkylamino e.g. aminoethylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, hydroxyC₁₋₆alkylamino e.g. hydroxyethylamino, imido, such as succinimido, nitro, cyano, amidino, hydroxyl (—OH), formyl [HC(O)—], carboxyl (—CO₂H), —CO₂Alk⁵ [where Alk⁵ is as defined above], e.g. —CO₂CH₃ or —CO₂CH₂CH₃, C₁₋₆alkanoyl e.g. acetyl, thiol (—SH), thioC₁₋₆alkyl, e.g. thiomethyl or thioethyl, —SC(=NH)NH₂, sulphonyl (—SO₃H), —SO₃R¹⁴, C₁₋₆alkylsulphinyl e.g. methylsulphinyl, C₁₋₆alkylsulphonyl, e.g. methylsulphonyl, aminosulphonyl (—SO₂NH₂), C₁₋₆alkylaminosulphonyl, e.g. methylaminosulphonyl or ethylaminosulphonyl, C₁₋₆dialkylaminosulphonyl, e.g. dimethylaminosulphonyl or diethylamino-sulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylamino-carbonyl, e.g. aminoethylaminocarbonyl, C₁₋₆dialkylaminoC₁₋₆alkylamino-carbonyl, e.g. diethylaminoethylaminocarbonyl, aminocarbonylamino, C₁₋₆alkylaminocarbonylamino, e.g. methylaminocarbonylamino or ethylamino-carbonylamino, C₁₋₆dialkylaminocarbonylamino, e.g. dimethylaminocarbonyl-amino or diethylaminocarbonylamino, C₁₋₆aikylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethyl-amino, —CONHC(=NH)NH₂, C₁₋₆alkylsulphonylamino, e.g. methylsulphonyl-amino or ethylsulphonylamino, C₁₋₆dialkylsulphonylamino, e.g. dimethyl-sulphonylamino or diethylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, C₁₋₆alkanoyl-amino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino or C₁₋₆alkoxycarbonylamino, e.g. methoxycarbonylamino, ethoxycarbonylamino or t-butoxycarbonylamino

Where desired, two R¹³ substituents may be linked together to form a cyclic group such as a cyclic ether, e.g. a C₁₋₆alkylenedioxy group such as methylenedioxy or ethylenedioxy.

It will be appreciated that where two or more R¹³ substituents are present, these need not necessarily be the same atoms and/or groups. In general, the substituent(s) may be present at any available ring position in the phenyl or pyridyl group represented by Ar.

In compounds of formula (1) derivatives of the carboxylic acid group R (—CO₂H) include carboxylic acid esters and amides. Particular esters and amides include —CO₂Alk⁵ [where Alk⁵ is as previously defined] and —CONR⁵R⁶ [where R⁵ and R⁶ is each either a hydrogen atom or an optionally substituted C₁₋₆alkyl and each may be the same or different] groups as defined herein. When R is a biostere of a carboxylic acid it may be for example a tetrazole or other acid such as phosphonic acid, phosphinic acid, sulphonic acid, sulphinic acid or boronic acid or an acylsulphonamide.

When, in compounds of formula (1), R³ is present as an atom or group —L¹(Alk^{l})ₜL²(R⁴)_{U} it may be present on any available carbon atom of the indole ring. When L¹ and/or L² is present in a group R³ as a linker atom or group it may be any linker atom or group as previously defined for L³. Alk¹, when present in the atom or group R³ may be any aliphatic or heteroaliphatic chain as defined hereinbefore.

When the groups R⁵ and R⁶ or R⁶ and R⁷ are present in R³ groups in compounds of formula (1) these groups may be joined, together with the N atom to which they are attached, to form a heterocyclic ring. Such heterocyclic rings may be optionally interrupted by a further heteroatom selected from —O—, —S— or —N(R⁵)—. Particular examples of such heterocyclic rings include piperidinyl, morpholinyl, thiomorpholinyl, pyrrolidinyl, imidazolidinyl and piperazinyl rings.

Examples of the R³ substituents represented by —L¹(Alk¹)ₜL²(R⁴)ᵤ when present in Ar¹ groups in compounds of the invention include atoms or groups —L¹Alk¹L²R⁴, —L¹Alk¹R⁴, —L¹R⁴ and —Alk¹R⁴ wherein L¹, Alk¹, L² and R⁴ are as defined above. Particular examples of such substituents include —L¹CH₂L²R⁴, —L¹CH(CH₃)L²R⁴, —L¹CH(CH₂)₂L²R⁴, —L¹CH₂R⁴, —L¹CH(CH₃)R⁴, —L¹(CH₂)₂R⁴, —CH₂R⁴, CH(CH₃)R⁴, —(CH₂)₂R⁴ and —R⁴ groups.

Thus the indole ring in compounds of formula (1) may be optionally substituted for example by one, two or three R³ atoms or groups where R³ is a halogen atom, e.g. fluorine, chlorine, bromine or iodine atom, or C₁₋₆alkyl, e.g. methyl, ethyl, n-propyl, i-propyl, n-butyl or t-butyl, C₁₋₆hydroxyalkyl, e.g. hydroxymethyl, hydroxyethyl or —C(OH)(CF₃)₂, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, haloC₁₋₆alkyl, e.g. —CF₃, —CHF₂, CH₂F, haloC₁₋₆alkoxy, e.g. —OCF₃, —OCHF₂, —OCH₂F, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, nitro, cyano, amidino, hydroxyl (—OH), formyl [HC(O)—], carboxyl (—CO₂H), —CO₂Alk³ [where Alk³ is as defined above for Alk⁵], C₁₋₆alkanoyl e.g. acetyl, thiol (—SH), thioC₁₋₆alkyl, e.g. thiomethyl or thioethyl, sulphonyl (—SO₃H), C₁₋₆alkylsulphonyl, e.g. methylsulphonyl, aminosulphonyl (—SO₂NH₂), C₁₋₆alkylaminosulphonyl, e.g. methylaminosulphonyl or ethylaminosulphonyl, C₁₋₆dialkylaminosulphonyl, e.g. dimethylaminosulphonyl or diethylaminosulphonyl, phenylaminosulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylaminocarbonyl, e.g. aminoethylaminocarbonyl, C₁₋₆dialkylamino-C₁₋₆alkylaminocarbonyl, e.g. diethylaminoethylaminocarbonyl, aminocarbonyl-amino, C₁₋₆alkylaminocarbonylamino, e.g. methylaminocarbonylamino or ethylaminocarbonylamino, C₁₋₆dialkylaminocarbonylamino, e.g. dimethylamino-carbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonyl-methylamino, C₁₋₆alkylsulphonylamino, e.g. methylsulphonylamino or ethylsulphonylamino, C₁₋₆dialkylsulphonylamino, e.g. dimethylsulphonylamino or diethylsulphonylamino, aminosulphonylamino (—NHSO₂NH₂), C₁₋₆alkylamino-sulphonylamino, e.g. methylaminosulphonylamino or ethylaminosulphonyl-amino, C₁₋₆dialkylaminosulphonylamino, e.g. dimethylaminosulphonylamino or diethylaminosulphonylamino, C₁₋₆alkanoylamino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxy-carbonylamino, e.g. methoxycarbonylamino, ethoxycarbonylamino or t-butoxycarbonylamino groups.

It will be understood that when two or three R³ groups are present in compounds of formula (1) these may be the same or different.

When the group R¹ is present in compounds of formula (1) as a C₁₋₆alkyl group it may be any C₁₋₆alkyl group as previously defined.

The presence of certain substituents in the compounds of formula (1) may enable salts of the compounds to be formed. Suitable salts include pharmaceutically acceptable salts, for example acid addition salts derived from inorganic or organic acids, and base addition salts derived from inorganic and organic bases.

Acid addition salts include hydrochlorides, hydrobromides, hydroiodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

Ar in compounds of the invention is preferably an optionally substituted phenyl or pyridyl group as hereinbefore defined.

One particular class of compounds of formula (1) is that wherein Alk is a —CH(R)(CH₂)ₘ group in which m is the integer 1.

A particularly useful group of compounds according to the invention has the formula (2):

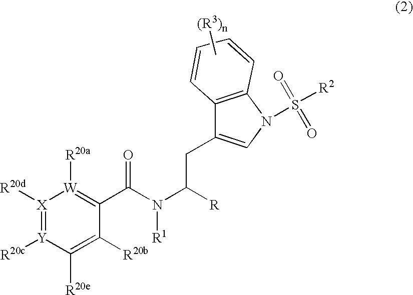

wherein:

W, X and Y is each a carbon atom or one of W, X and Y is a nitrogen atom and the others are carbon atoms;

R^{20a}, R^{20b}, R^{20c}, R^{20d} and R^{20e} are each a hydrogen atom or an atom or group

R¹³ as previously defined;

R, R¹, R², R³ and n are as previously defined;

provided that when one of W, X and Y is a nitrogen atom it is not substituted by R^{20a}, R^{20d} or R^{20c} respectively;

and the salts, solvates, hydrates and N-oxides thereof.

R¹ in compounds of the invention is in particular a hydrogen atom.

R in compounds of the invention is preferably a carboxylic acid (—CO₂H) or a carboxylic acid ester (—CO₂Alk⁵). Particularly useful Alk⁵ groups include alkyl groups, especially methyl, ethyl and i-propyl groups. Most preferably R is a carboxylic acid (—CO₂H).

R² in compounds of the invention is preferably an optionally substituted C₁₋₆alkyl group. Most preferably R² is an optionally substituted methyl group, especially a methyl or ethyl group. Preferred optional substituents include one, two or three halogen atoms, especially fluorine or chlorine atoms

In general in compounds of formula (1) and (2) n is zero or the integer 1 or 2. When present R³ is preferably a halogen atom or an C₁₋₆alkyl, haloC₁₋₆alkyl, C₁₋₆alkoxy (—OR⁵), haloC₁₋₆alkoxy, hydroxy, nitro, cyano or —NR⁵R⁶ group. Particularly useful halogen atoms include fluorine and chlorine atoms. Particularly useful C₁₋₆alkyl groups include methyl and ethyl groups, particularly useful haloC₁₋₆alkyl groups include —CF₃, particularly useful C₁₋₆alkoxy groups include methoxy and ethoxy groups and particularly useful haloC₁₋₆.₆alkoxy groups include —OCF₃ groups. Particularly useful —NR⁵R⁶ groups include —NHCH₃ and —N(CH₃)₂ groups.

In compounds of formula (2) R^{20e} is preferably a hydrogen or halogen atom, especially a fluorine or chlorine atom. Most preferably R^{20e} is a hydrogen atom.

In another preferred class of compounds of formulae (1) and (2) W, X and Y is each an optionally substituted CH group. In one preferred group of compounds of this class R^{20a}, R^{20c} and R20d is each a hydrogen atom and R^{20e} is as just defined. R^{20b} in this group of compounds is preferably a halogen atom, especially a fluorine or chlorine atom, or a C₁₋₆alkyl, especially methyl or ethyl, haloC₁₋₆alkyl, especially trifluoromethyl, C₁₋₆alkoxy, especially methoxy or ethoxy, haloC₁₋₆alkoxy, especially trifluoromethoxy or nitro group. Most preferably R^{20b} is a chlorine atom. In another preferred group of compounds of this class R^{20c} and R^{20d} is each a hydrogen atom and R^{20e} is as just defined. R^{20a} and R^{20b} in this group of compounds is each preferably a halogen atom, especially a fluorine or chlorine atom, or a C₁₋₆alkyl, especially methyl or ethyl, haloC₁₋₆alkyl, especially trifluoromethyl, C₁₋₆alkoxy, especially methoxy or ethoxy, haloC₁₋₆alkoxy, especially trifluromethoxy or nitro group. Most preferably R^{20a} and R^{20b} is each a chlorine atom. In another preferred group of compounds of this class R^{20d} is a hydrogen atom and R^{20e} is as just defined. In this group of compounds R^{20a} and R^{20b} is each preferably an atom or group selected from a halogen atom, especially a fluorine or chlorine atom, or a C₁₋₆alkyl, especially methyl or ethyl, haloC₁₋₆alkyl, especially trifluoromethyl, C₁₋₆alkoxy, especially methoxy or ethoxy, haloC₁₋₆alkoxy, especially trifluoromethoxy or nitro group. In this group of compounds R^{20c} is preferably a group of formula —CONH Alk²R^{13a} or —CSNHAlk²R^{13a} as hereinbefore generally defined. In this group Alk² is preferably a —CH₂— or —CH₂CH₂— chain and R^{13a} is preferably an optionally phenyl, thienyl, furanyl, pyridyl or pyrimidinyl group where preferred optional substituents include those preferred atoms and groups as hereinbefore describe in relation to R^{3.}

In another preferred class of compounds of formulae (1) and (2) W and X is each an optionally substituted CH group and Y is a nitrogen atom. In this class of compounds R^{20c} is absent. In one preferred group of compounds of this class R^{20a} and R^{20d} is each a hydrogen atom and R^{20e} is as just defined. R^{20b} in this group of compounds is preferably a halogen atom, especially a fluorine or chlorine atom, or a C₁₋₆alkyl, especially methyl or ethyl, haloC₁₋₆alkyl, especially trifluoromethyl, C₁₋₆alkoxy, especially methoxy or ethoxy, haloC₁₋₆alkoxy, especially trifluoromethoxy or nitro group. Most preferably R^{20b} is a chlorine atom. In another preferred group of compounds of this class R^{20d} is a hydrogen atom and R^{20e} is as just defined. R^{20a} and R^{20b} in this group of compounds is each preferably a halogen atom, especially a fluorine or chlorine atom, or a C₁₋₆alkyl, especially methyl or ethyl, haloC₁₋₆alkyl, especially trifluoromethyl, C₁₋₆alkoxy, especially methoxy or ethoxy, haloC₁₋₆alkoxy, especially trifluoromethoxy or nitro group. Most preferably R^{20a} and R^{20b} is each a chlorine atom.

In another preferred class of compounds of formulae (1) and (2) X and Y is each an optionally substituted CH group and W is a nitrogen atom. In this class of compounds R^{20a} is absent, R^{20c} and R^{20d} is each preferably a hydrogen atom and R^{20b} is preferably a halogen atom, especially a fluorine or chlorine atom, or a methyl, trifluromethyl or nitro group. Most preferably R^{20b} is a chlorine atom.

In another preferred class of compounds of formulae (1) and (2) W and Y is each an optionally substituted CH group and X is a nitrogen atom. In this class of compounds R^{20d} is absent, R^{20a} is preferably a hydrogen or halogen, especially fluorine or chlorine atom, or a C₁₋₆alkyl, especially methyl or ethyl, haloC₁₋₆alkyl, especially trifluromethyl, C₁₋₆alkoxy, especially methoxy or ethoxy, haloC₁₋₆alkoxy, especially trifluromethoxy or nitro group, R^{20b} is preferably a hydrogen or halogen atom, especially a fluorine or chlorine atom, or a C₁₋₆alkyl, especially methyl or ethyl, haloC₁₋₆alkyl, especially trifluromethyl, C₁₋₆alkoxy, especially methoxy or ethoxy, haloC₁₋₆alkoxy, especially trifluromethoxy or nitro group and R^{20c} is preferably a hydrogen or halogen atom, especially a fluorine or chlorine atom, or a C₁₋₆alkyl, especially methyl or ethyl, haloC₁₋₆alkyl, especially trifluromethyl, C₁₋₆alkoxy, especially methoxy or ethoxy, haloC₁₋₆alkoxy, especially trifluromethoxy or nitro group and R^{20e} is as just defined. Most preferably R^{20a} and R^{20c} is each a hydrogen atom, R^{20b} is a chlorine atom and R^{20e} is as just defined or R^{20a} is a chlorine atom and R^{20b} and R^{20c} is each a hydrogen atom and R^{20e} is as just defined or R^{20a} is a chlorine atom, R^{20b} is a hydrogen atom and R^{20c} is a chlorine atom or methyl group.

Particularly useful compounds of the invention include:

(2S)-2-[(3,5-dichloropyridine-4-carbonyl)-amino]-3-(1-methanesulfonyl-1H-indol-3-yl)-propionic acid;

(2S)-2-(2,6-dichlorobenzoylamino)-3-(1 -methanesulfonyl-1H-indol-3-yl)-propionic acid;

(2S)-2-[(2-chloropyridine-3-carbonyl)-amino]-3-(1-methanesulfonyl-1 H-indol-3-yl)-propionic acid;

2-[2-chloro-4-(3-hydroxy-benzylcarbamoyl)-benzoylamino]-3-( 1-methanesulfonyl-1H-indol-3-yl)-propionic acid;

2-(2,6-dichloro-benzoylamino)-3-(1-methanesulfonyl-1H-indol-3-yl)-propionic acid;

2-[(3,5-dichloro-pyridine-4-carbonyl)-amino]-3-(1-methanesulfonyl-1H-indol-3-yl)-propionic acid;

and the salts, solvates, hydrates and N-oxides thereof.

Compounds according to the invention are potent inhibitors of LFA-1 binding to cellular adhesion molecules, particularly ICAM-1, -2 or -3. The ability of the compounds to act in this way may be simply determined by employing tests such as those described in the Examples hereinafter.

The compounds are of use in modulating LFA-1 mediated cell signalling and in particular are of use in the prophylaxis and treatment of diseases or disorders involving inappropriate migration of cells. The invention extends to such a use and to the use of the compounds of formula (1) for the manufacture of a medicament for treating such diseases and disorders. Particular diseases include inflammatory diseases and immune disorders.

Particular uses to which the compounds of the invention may be put include the treatment or inhibition of acute or chronic inflammatory diseases or disorders or autoimmune diseases e.g. rheumatoid arthritis, systemis lupus erythematosus, hashimoto's thyroidis, multiple sclerosis, myasthenia gravis, diabetes type 1 and uveitis, cutaneous manifestations of immunologically mediated illness such as inflammatory and hyperproliferative skin diseases (e.g. psoriasis, atopic dermatitis, alopecia aerata, allergic contact dermatitis, irritant contact dermatitis, eczematous dermatitis, seborrhoeic dermatitis, lichen planus, pemphigus, bullous pemphigoid, epidermolysis bullosa, urticaria, angioedemas, vasculitides, erthema multiforme, cutaneous eosinophilias, lupus erythematosus, acne, granuloma annulare, pyoderma gangrenosum, sun burns and toxic epidermal necrolysis), inflammatory bowel disease, opthalmic inflammatory diseases or immune-mediated conditions of the eye, such as auto-immune diseases (e.g. keratoplasty and allergic keratitis), allergic conditions (e.g. vernal conjunctivitis), inflammatory conditions and corneal transplants. Compounds of formula (1) are further useful for the treatment and/or prevention of ischemia-reperfusion injury e.g myocardial infarction, stroke, gut ischemia, renal failure, graft vs. host and host vs. graft rejection, renal failure or hemorrhage shock, and infective diseases such as septic shock, adult respiratory distress syndrome or traumatic shock.

Especially preferred uses to which compounds of the invention may be put include the tretament or inhibition of cutaneous manifestations of immunologically mediated illness such as inflammatory and hyperproliferative skin diseases (e.g. psoriasis, atopic dermatitis, alopecia aerata, allergic contact dermatitis, irritant contact dermatitis, eczematous dermatitis, seborrhoeic dermatitis, lichen planus, pemphigus, bullous pemphigoid, epidermolysis bullosa, urticaria, angioedemas, vasculitides, erthema multiforme, cutaneous eosinophilias, lupus erythematosus, acne, granuloma annulare, pyoderma gangrenosum, sun burns and toxic epidermal necrolysis), most especially psoriasis, atopic dermatitis, allergic contact dermatitis, irritant contact dermatitis, eczematous dermatitis or seborrhoeic dermatitis.

For the prophylaxis or treatment of disease the compounds according to the invention may be administered as pharmaceutical compositions, and according to a further aspect of the invention we provide a pharmaceutical composition which comprises a compound of formula (1) together with one or more pharmaceutically acceptable carriers, excipients or diluents.

Pharmaceutical compositions according to the invention may take a form suitable for oral, buccal, parenteral, nasal, topical, vaginal or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in conventional manner.

The compounds for formula (1) may be formulated for parenteral administration by injection e.g. by bolus injection or infusion. Formulations for injection may be presented in unit dosage form, e.g. in glass ampoule or multi dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use. For particle mediated administration the compounds of formula (1) may be coated on particles such as microscopic gold particles.

In addition to the formulations described above, the compounds of formula (1) may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichloro-fluoromethane, dichlorotetrafluoroethane, carbon dioxide or other suitable gas or mixture of gases.

For vaginal or rectal administration the compounds of formula (1) may be formulated as a suppository. These formulations may be prepared by mixing the active ingredient with a suitable non-irritating excipient which is a solid at room temperature but liquid at the body temerature. Such materials include for example cocoa butter and polyethylene glycols.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg e.g. around 0.01 mg/kg to 40 mg/kg body weight for oral or buccal administration, from around 10 ng/kg to 50 mg/kg body weight for parenteral administration and around 0.05 mg to around 1000 mg e.g. around 0.5 mg to around 1000 mg for nasal administration or administration by inhalation or insufflation.

The compounds of the invention may be prepared by a number of processes as generally described below and more specifically in the Examples hereinafter. Many of the reactions described are well-known standard synthetic methods which may be applied to a variety of compounds and as such can be used not only to generate compounds of the invention, but also where necessary the intermediates thereto.

In the following process description, the symbols R¹, Alk, m, Alk⁵, R³, n, R², X, Ar, L², L³, R¹⁴ and R⁶ when used in the formulae depicted are to be understood to represent those groups described above in relation to formula (1) unless otherwise indicated. In the reactions described below, it may be necessary to protect reactive functional groups, for example hydroxy, amino, thio or carboxy groups, where these are desired in the final product, to avoid their unwanted participation in the reactions. Conventional protecting groups may be used in accordance with standard practice [see, for example, Green, T. W. in “Protective Groups in Organic Synthesis”, John Wiley and Sons, (1999) and the examples herein]. In some instances, deprotection may be the final step in the synthesis of a compound of formula (1) and the processes according to the invention described hereinafter are to be understood to extend to such removal of protecting groups.

Thus according to a further aspect of the invention a compound of formula (1) in which Alk is a chain

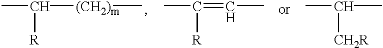

in which m is zero or the integer for 2 and R is a carboxylic acid (—CO₂H) may be prepared by the reactions illustrated in Scheme (1):

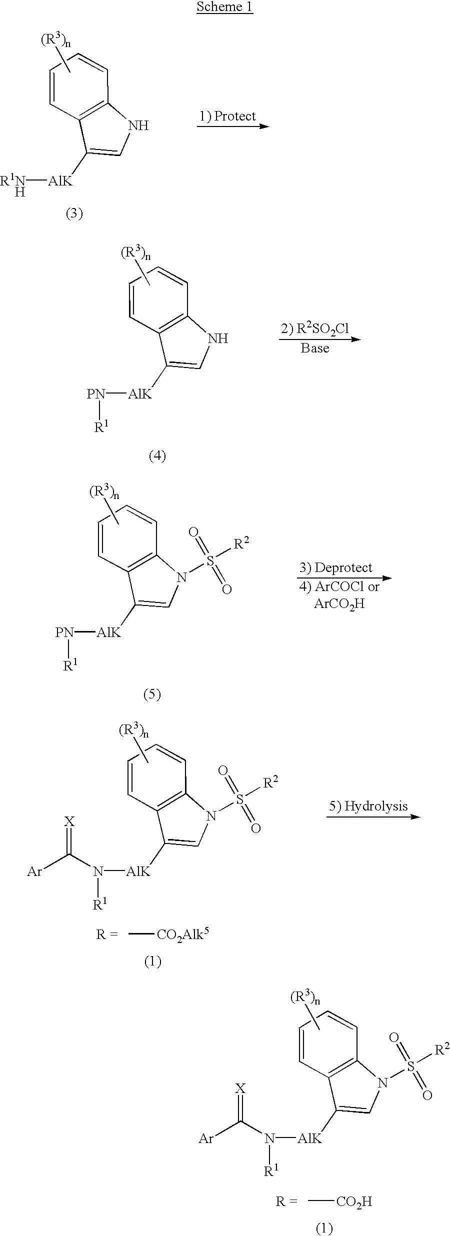

An amino acid analogue of formula (3) in which R is a carboxylic acid ester (—CO₂Alk⁵) in which for example Alk⁵ is an alkyl group such as a methyl or ethyl group may be N-protected to yield an protected intermediate of formula (4) (P=protecting group) by any standard method for protection of amino acids such as those described in Greene (ibid), Bodansky, M. [Principles of Peptide Synthesis, 2^{nd} ed., Springer-Verlag, Berlin (1993)] or Kocienski, P. J. [Protecting Groups, Thieme, Stuttgart (1994)]. Thus for example an amino acid intermediate of formula (3) may be N-protected with a t-butyloxycarbonyl group (P =BOC i.e. (CH₃)₃COC(O)—) by reaction with an anhydride ( [(CH₃)₃COC(O)]₂O) or a chloroformate [(CH₃)₃CC(O)Cl] in an organic solvent such as a halogenated hydrocarbon e.g. dichloromethane, an ether e.g. a cyclic ether such as tetrahydrofuran or dioxane, a nitrile e.g. acetonitrile or an amide e.g. a substituted amide such as dimethylformamide optionally in the presence of water and a base such as a carbonate e.g. caesium or potassium carbonate or sodium hydrogen carbonate, a hydroxide e.g. sodium or potassium hydroxide or an amine e.g. triethylamine or N-methylmorpholine at a temperature from about 0° C. to ambient temperature.

Amino acid intermediates of formula (3) are commercially available or may be formed by methods known in the literature, for example the methods of Cook, J. M. et al (Tetrahedron Lett., 1995, 7411-7414 and J. Org. Chem. 1997, 62, 7447-7456), Thiruvikraman, S. V. and Sakagami, Y. (Tetrahedron Lett., 1988, 2339-2342), Li, M. and Johnson, M. E. (Tetrahedron Lett., 1994, 6255-6258), Balsamini, C. et al (Synthesis, 1995, 370-372), Horwell, D. C. et al (J. Org. Chem., 1994, 59, 4418-4423), Ma, C et al (Tetrahedron Lett., 2000, 2781-2785), Morales-Rios, M. S. et al (Heterocycles, 1996, 43, 1483-1496) and Gademann, K. et al (Angew Chem Int Ed, 1999, 38,1223-1226).

A sulphonamide derivative of formula (5) may be formed by reaction of a protected amino acid derivative of formula (4) with a sulphonating agent such as a sulphonyl halide (R²SO₂Hal), for example methanesulphonyl chloride or an anhydride [(R²SO₂)₂] such as trifluoromethanesulphonic anhydride in an organic solvent such as a halogenated hydrocarbon e.g. dichloromethane or an ether e.g. a cyclic ether such as tetrahydrofuran or dioxane optionally in the presence of a base such as an amine e.g. triethylamine, N-methylmorpholine or pyridine or a hydroxide e.g. sodium or potassium hydroxide at a temperature from about −78° C. to ambient temperature.

A sulphonamide of formula (5) may be N-deprotected by using any standard deprotection conditions depending on the nature of the protecting group P. Thus, for example, when P is a BOC group deprotection may be accomplished with an acid, for example an inorganic acid such as hydrochloric acid or an organic acid such as trifluoroacetic acid, or a silane such as chlorotrimethylsilane in the presence of phenol optionally in an organic solvent such as a halogenated hydrocarbon e.g. dichloromethane at a temperature from around 0° C. to around ambient temperature.

Subsequent to this deprotection step an amide of formula (1) (X=O, R=—CO₂Alk⁵) may be formed by coupling the deprotected amine derived from a compound of formula (5) with an acid of formula (6):

ArCO₂H (6)

or an active derivative thereof.

Active derivatives of acids of formula (6) include anhydrides, esters and halides. Particular esters include pentafluorophenyl or succinyl esters. Particular halides include chlorides.

The coupling reaction may be performed using standard conditions for reactions of this type. Thus for example the reaction may be carried out in a solvent, for example an inert solvent such as an amide e.g. a substituted amide such as dimethylformamide, an ether e.g. a cyclic ether such as tetrahydrofuran, or a halogenated hydrocarbon such as dichloromethane, at a low temperature e.g. around −30° C. to around ambient temperature, optionally in the presence of a base e.g. an organic base such as amine e.g. triethylamine or pyridine or dimethylaminopyridine, or a cyclic amine such as N-methylmorpholine.

When an acid of formula (6) is used, the reaction may additionally be performed in the presence of a condensing agent, for example a diimide such as 1-(3-dimethylaminopropyl)-3-ethylcarbodiimide or N,N′-dicyclohexyl-carbodimide, advantageously in the presence of a catalyst compound such as a N-hydroxy compound e.g. a N-hydroxytriazole such as 1-hydroxybenzotriazole. Alternatively, the acid may be reacted with a chloroformate, for example ethyl chloroformate, prior to reaction with the amine of formula (6).

Thioamides of formula (1) (X=S, R=—CO₂Alk⁵) may be prepared from amides of formula (1) by treatment with a thiation reagent, such as Lawesson's reagent, in an anhydrous solvent, for example a cyclic ether such as tetrahydrofuran, at an elevated temperature such as the reflux temperature.

A compound of formula (1) (R=—CO₂H) may be obtained from an ester of formula (1) (R=—CO₂Alk⁵) by hydrolysis. The hydrolysis may be performed using either an acid or a base depending on the nature of Alk⁵, for example an organic acid such as trifluoroacetic acid optionally in an organic solvent such as a halogenated hydrocarbon e.g. dichloromethane, or an inorganic base such as sodium, lithium or potassium hydroxide optionally in an aqueous organic solvent such as an amide e.g. a substituted amide such as dimethylformamide, an ether, e.g. a cyclic ether such as tetrahydrofuran or dioxane or an alcohol, e.g. methanol at around ambient temperature to 60° C. Where desired, mixtures of such solvents may be used.

Where in the general processes described above intermediates such as esters of formulae (3), acids of formula ArCO₂H and sulphonyl chlorides of formula R²SO₂Cl are not available commercially or known in the literature, they may be readily obtained from simpler known compounds by one or more standard synthetic methods employing substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other intermediates and in particular compounds of formula (1) where appropriate functional groups exist in these compounds. Particular examples of such methods are given in the Examples hereinafter.

Thus intermediates of formula (3) and any other intermediates described herein required to obtain compounds of formula (1) may be prepared by methods known to those skilled in the art following procedures set forth in references such as Rodd's Chemistry of Carbon Compounds, Volumes 1-15 and Supplementals (Elsevier Science Publishers, 1989), Fieser and Fieser's Reagents for Organic Synthesis, Volumes 1-19 (John Wiley and Sons, 1999), Comprehensive Heterocyclic Chemistry, Ed. Katritzky et al, Volumes 1-8, 1984 and Volumes 1-11, 1994 (Pergamon), Comprehensive Organic Functional Group Transformations, Ed. Katritzky et al, Volumes 1-7, 1995 (Pergamon), Comprehensive Organic Synthesis, Ed. Trost and Flemming, Volumes 1-9, (Pergamon, 1991), Encyclopedia of Reagents for Organic Synthesis Ed. Paquette, Volumes 1-8 (John Wiley and Sons, 1995), Larock's Comprehensive Organic Transformations (VCH Publishers Inc., 1989) and March's Advanced Organic Chemistry (John Wiley and Sons, 1992)

For example sulphonyl chlorides of formula R²SO₂Cl may be formed by reaction of Grignard reagents of formula R²MgHal where Hal is a halogen atom such as a chlorine, bromine or iodine atom or lithium reagents of formula R²Li with sulfuryl chloride in an inert solvent such as a hydrocarbon e.g. pentane or hexane or an aromatic hydrocarbon e.g. toluene at a low temperature, for example about −65 to about −20° C.

Sulphonyl halides such as sulphonyl chlorides of formula R2SO₂Cl may also be formed from sulphonic acids of formula R²SO₂H by reaction with a halogenating agent such as a thionyl halide e.g. thionyl chloride, a phosphorous trihalide such as phosphorous trichloride or a phosphorous pentahalide such as phosphorous pentachloride optionally in an inert solvent such as an aromatic hydrocarbon e.g. toluene or a chlorinated hydrocarbon e.g. dichloromethane at a temperature from about 0° C. to the reflux temperature.

Aromatic halogen substituents in the compounds may be subjected to halogen-metal exchange with a base, for example a lithium base such as n-butyl or t-butyl lithium, optionally at a low temperature, e.g. around −78° C., in a solvent such as tetrahydrofuran and then quenched with an electrophile to introduce a desired substituent. Thus, for example, a formyl group may be introduced by using dimethylformamide as the electrophile, a thiomethyl group may be introduced by using dimethyldisulphide as the electrophile, an alcohol group may be introduced by using an aldehyde as electrophile and an acid may be introduced by using carbon dioxide as electrophile. Aromatic acids of formula ArCO₂H may also be generated by quenching Grignard reagents of formula ArMgHal with carbon dioxide. Aromatic acids of formula ArCO₂H generated by this method may be converted to activated derivatives, e.g. acid halides by the methods just described for the conversion of sulphonic acids to sulphonyl halides.

Compounds of the invention and intermediates thereto such as compounds of formulae (3), (4), (5), ArCO₂H and R²SO₂Cl may be prepared by alkylation, arylation or heteroarylation. For example, compounds containing a —L¹H group (where L^{l} is a linker atom or group) may be treated with an alkylating agent (R⁴)_{U}L²(Alk¹)ₜZ² in which Z² is a leaving atom or group such as a halogen atom, e.g. a fluorine, chlorine, bromine or iodine atom or a sulphonyloxy group such as an alkylsulphonyloxy, e.g. trifluoromethylsulphonyloxy or arylsulphonyloxy, e.g. p-toluenesulphonyloxy group.

The reaction may be carried out in the presence of a base such as a carbonate, e.g. caesium or potassium carbonate, an alkoxide, e.g. potassium t-butoxide, or a hydride, e.g. sodium hydride, in a dipolar aprotic solvent such as an amide, e.g. a substituted amide such as dimethylformamide or an ether, e.g. a cyclic ether such as tetrahydrofuran.

In another example, compounds containing a —L¹H group as defined above may be functionalised by acylation or thioacylation, for example by reaction with the alkylating agents just described but in which Z² is replaced by a —C(O)Z³, C(S)Z³, —N(R¹⁷)COZ³or —N(R¹⁷)C(S)Z³ group in which Z³ is a leaving atom or group as described for Z². The reaction may be performed in the presence of a base, such as a hydride, e.g. sodium hydride or an amine, e.g. triethylamine or N-methylmorpholine, in a solvent such as a halogenated hydrocarbon, e.g. dichloromethane or carbon tetrachloride or an amide, e.g. dimethylformamide, at for example ambient temperature. Alternatively, the acylation may be carried out under the same conditions with an acid (for example one of the alkylating agents described above in which Z² is replaced by a —CO₂H group) in the presence of a condensing agent, for example a diimide such as 1-(3-dimethylaminopropyl)-3-ethylcarbodiimide or N,N′-dicyclohexylcarbodiimide, or a benzotriazole such as [O-(7-azabenzo-triazol-1-yl)-1,1,3,3-tetramethyluronium]hexafluorophosphate advantageously in the presence of a catalyst such as a N-hydroxy compound e.g. a N-hydroxytriazole such as 1-hydroxybenzotriazole. Alternatively the acid may be reacted with a chloroformate, for example ethylchloroformate, prior to the desired acylation reaction

In a further example compounds may be obtained by sulphonylation of a compound containing an —OH group by reaction with one of the above alkylating agents but in which Z² is replaced by a —S(O)Hal or —SO₂Hal group [in which Hal is a halogen atom such as chlorine atom] in the presence of a base, for example an inorganic base such as sodium hydride in a solvent such as an amide, e.g. a substituted amide such as dimethylformamide at for example ambient temperature.

In another example, compounds containing a —L²H group as defined above may be coupled with one of the alkylation agents just described but in which Z² is replaced by an —OH group in a solvent such as tetrahydrofuran in the presence of a phosphine, e.g. triphenylphosphine and an activator such as diethyl, diisopropyl- or dimethylazodicarboxylate.

Ester groups such as —CO₂Alk⁵ and —CO₂R⁹ in the compound of formula (1) and intermediates thereto may be converted to the corresponding acid [—CO₂H] by acid- or base-catalysed hydrolysis depending on the nature of the group Alk⁵. Acid- or base-catalysed hydrolysis may be achieved for example by treatment with an organic or inorganic acid, e.g. trifluoroacetic acid in an organic solvent e.g. dichloromethane or a mineral acid such as hydrochloric acid in a solvent such as dioxan or an alkali metal hydroxide, e.g. lithium hydroxide in an aqueous alcohol, e.g. aqueous methanol.

In a further example, —OR¹⁴ [where R¹⁴ represents an alkyl group such as methyl group] in compounds of formula (1) and intermediates thereto may be cleaved to the corresponding alcohol —OH by reaction with boron tribromide in a solvent such as a halogenated hydrocarbon, e.g. dichloromethane at a low temperature, e.g. around −78° C.

Alcohol [—OH] groups may also be obtained by hydrogenation of a corresponding —OCH₂R²⁵ group (where R²⁵ is an aryl group) using a metal catalyst, for example palladium on a support such as carbon in a solvent such as ethanol in the presence of ammonium formate, cyclohexadiene or hydrogen, from around ambient to the reflux temperature. In another example, —OH groups may be generated from the corresponding ester [e.g. —CO₂AlK⁵] or aldehyde [—CHO] by reduction, using for example a complex metal hydride such as lithium aluminium hydride or sodium borohydride in a solvent such as methanol.

In another example, alcohol —OH groups in the compounds may be converted to a corresponding —OR¹⁴ group by coupling with a reagent R¹⁴OH in a solvent such as tetrahydrofuran in the presence of a phosphine, e.g. triphenylphosphine and an activator such as diethyl-, diisopropyl-, or dimethylazodicarboxylate.

Aminosulphonylamino [—NHSO₂NH₂] groups in the compounds may be obtained, in another example, by reaction of a corresponding amine [—NH₂] with sulphamide in the presence of an organic base such as pyridine at an elevated temperature, e.g. the reflux temperature.

In another example compounds containing a —NHCSR⁶ or —CSNHR⁶ group may be prepared by treating a corresponding compound containing a —NHCOR⁶ or —CONHR⁶ group with a thiation reagent, such as Lawesson's Reagent or P₂S₅, in an anhydrous solvent, for example a cyclic ether such as tetrahydrofuran, at an elevated temperature such as the reflux temperature.

In a further example amine (—NH₂) groups may be alkylated using a reductive alkylation process employing an aldehyde and a reducing agent. Suitable reducing agents include borohydrides for example sodium triacetoxyborohyride or sodium cyanoborohydride. The reduction may be carried out in a solvent such as a halogenated hydrocarbon, e.g. dichloromethane, a ketone such as acetone, or an alcohol, e.g. ethanol, where necessary in the presence of an acid such as acetic acid at around ambient temperature. Alternatively, the amine and aldehyde may be initially reacted in a solvent such as an aromatic hydrocarbon e.g. toluene and then subjected to hydrogenation in the presence of a metal catalyst, for example palladium on a support such as carbon, in a solvent such as an alcohol, e.g. ethanol.

In a further example, amine [—NH₂] groups in compounds of formula (1) and intermediates thereto may be obtained by hydrolysis from a corresponding imide by reaction with hydrazine in a solvent such as an alcohol, e.g. ethanol at ambient temperature.

In another example, a nitro [—NO₂] group may be reduced to an amine [—NH₂], for example by catalytic hydrogenation using for example hydrogen in the presence of a metal catalyst, for example palladium on a support such as carbon in a solvent such as an ether, e.g. tetrahydrofuran or an alcohol e.g. methanol, or by chemical reduction using for example a metal, e.g. tin or iron, in the presence of an acid such as hydrochloric acid.

In a further example amine (—CH₂NH₂) groups in compounds of formula (1) and intermediates thereto may be obtained by reduction of nitrites (—CN), for example by catalytic hydrogenation using for example hydrogen in the presence of a metal catalyst, for example palladium on a support such as carbon, or Raney® nickel, in a solvent such as an ether e.g. a cyclic ether such as tetrahydrofuran or an alcohol e.g. methanol or ethanol, optionally in the presence of ammonia solution at a temperature from ambient to the reflux temperature, or by chemical reduction using for example a metal hydride e.g. lithium aluminium hydride, in a solvent such as an ether e.g. a cyclic ether such as tetrahydrofuran, at a temperature from 0° C. to the reflux temperature.

In another example, sulphur atoms in the compounds, for example when present in a group L¹ may be oxidised to the corresponding sulphoxide or sulphone using an oxidising agent such as a peroxy acid, e.g. 3-chloroperoxybenzoic acid, in an inert solvent such as a halogenated hydrocarbon, e.g. dichloromethane, at around ambient temperature.

In a further example N-oxides of compounds of formula (1) may in general be prepared for example by oxidation of the corresponding nitrogen base using an oxidising agent such as hydrogen peroxide in the presence of an acid such as acetic acid, at an elevated temperature, for example around 70° C. to 80° C., or alternatively by reaction with a peracid such as peracetic acid or m-chloroperoxybenzoic acid in a solvent,such as a halogenated hydrocarbon e.g. dichloromethane or an alcohol e.g. tert-butanol at a temperature from the ambient temperature to the reflux temperature.

Salts of compounds of formula (1) may be prepared by reaction of a compound of formula (1) with an appropriate acid or base in a suitable solvent or mixture of solvents e.g. an organic solvent such as an ether e.g. diethylether, or an alcohol, e.g. ethanol using conventional procedures.

Where it is desired to obtain a particular enantiomer of a compound of formula (1) this may be produced from a corresponding mixture of enantiomers using any suitable conventional procedure for resolving enantiomers.

Thus for example diastereomeric derivatives, e.g. salts, may be produced by reaction of a mixture of enantiomers of formula (1) e.g. a racemate, and an appropriate chiral compound, e.g. a chiral base. The diastereomers may then be separated by any convenient means, for example by crystallisation and the desired enantiomer recovered, e.g. by treatment with an acid in the instance where the diastereomer is a salt.

In another resolution process a racemate of formula (1) may be separated using chiral High Performance Liquid Chromatography. Alternatively, if desired a particular enantiomer may be obtained by using an appropriate chiral intermediate in one of the processes described above.

Chromatography, recrystallisation and other conventional separation procedures may also be used with intermediates or final products where it is desired to obtain a particular geometric isomer of the invention.

The following Examples illustrate the invention. All temperatures are in 0° C. The following abbreviations are used:

| | |
|---|---|
| THF-tetrahydrofuran; | boc-butoxycarbonyl |
| DMF-dimethylformamide; | DMSO-dimethyl suiphoxide; |
| DOM-dichloromethane; | TFA-trifluoroacetic acid; |
| MeOH-methanol; | EtOH-ethanol |
| EtOAc-ethyl acetate. | nBuLi-n-butyllithium |
| RT-room temperature | Et₃N-triethylamine |
| sat.-saturated | HOBT-1-hydroxybenzotriazole hydrate |
| NMM-4-methylmorpholine | ether-diethyl ether |
| DIEA-dilsopropylethylamine | t.l.c.-thin layer chromatography |

EDC-1-[3-(dimethylamino)propyl]-3-ethylcarbodiimide hydrochloride
DBU-1,8-diazabicyclo[5.4.0]undec-7-ene
PS-Trisamine-Tris-(2-aminoethyl)amine polystyrene
MP-TsOH-Macroporous polystyrene sulfonic acid

All NMRs were obtained at 300 MHz unless otherwise indicated.

### Intermediate 1

(2S)-2-tert-Butoxycarbonylamino-3-(1H-indol-3-yl)-propionic acid methyl ester

A solution of di-tert-butyl dicarbonate (8.72 g, 40.0 mmol) in dioxane (200 ml) was added to a suspension of L-tryptophan methyl ester hydrochloride (10.18 g, 40.0 mmol) and sodium hydrogen carbonate (16.8 g, 200 mmol) in water (150 ml). The mixture was stirred at RT for 5 h. The dioxane was removed in vacuo, water (50 ml) added to the residue which was then extracted with EtOAc (300 ml+100 ml). The combined organics were dried (Na₂SO₄) and concentrated in vacuo to give the title compound as a white solid (12.54 g, 98%). δ_{H} (CDCl₃) 8.11 (1H, br s), 7.55 (1 H, d, J 7.9 Hz), 7.35 (1H, d, J8.1 Hz), 7.22-7.10 (2H, m), 7.00 (1H, d, J2.3 Hz), 5.07, (1H, br m), 4.67 (1H, br m), 3.68 (3H, s), 3.30 (2H, m) and 1.53 (9H, br s); m/z (ES⁺) 341.1 (Mna⁺).

### Intermediate 2

(2S)-2-tert-Butoxycarbonylamino-3-(1 -methanesulfonyl-1H-indol-3-yl)-propionic acid methyl ester

Methanesulfonyl chloride (1.46 ml, 18.85 mmol) was added over 15 min to a solution of Intermediate 1 (6.0 g, 18.85 mmol) and Et₃N (5.25 ml, 37.7 mmol) in CH₂Cl₂ (100 ml). After 2 h at RT more methanesulfonyl chloride (1.46 ml, 18.85 mmol) was added. After a further 18 h more triethylamine (2.8 ml) and methanesulfonyl chloride (1.46 ml, 18.85 mmol) were added. After a further 4 h the solution was washed with 1 M HCl (100 ml) and sat. NaHCO₃ (100 ml), dried (Na₂SO₄) and concentrated in vacuo. Purification by column chromatography (SiO₂; 1-2% acetone in DCM) gave the title compound as a colourless gum (2.6 g, 35%). δ_{H} (CDCl₃) 7.90 (1 H, d, J 7.5 Hz), 7.58 (1 H, d, J 7.1 Hz), 7.38-7.31 (2H, m), 7.24 (1H, s), 4.99 (1 H, br m), 4.66 (1 H, br m), 3.71 (3H, s), 3.39-3.36 (1H, m), 3.29-3.26 (1H, m), 3.05 (3H, s), 1.43 (9H, s); m/z (ES⁺) 419.2 (Mna⁺).

### Intermediate 3

(2S)-2-Amino-3-(1-methanesulfonyl-1H-indol-3-yl)-propionic acid methyl ester hydrochloride

Intermediate 2 (2.60 g, 6.54 mmol) was treated with 2.6 M HCl in EtOAc (75 ml) for 4 h at RT. Volatiles were removed in vacuo and the residue triturated with Et₂O (50 ml). The pale pink-purple solid was filtered off, washed with Et₂O and dried to give the title compound (1.60 g, 74%). δ_{H} (d₆-DMSO) 8.53 (2H, br s, NH₂), 7.63 (1H, d, J 8.0 Hz), 7.45 (1H, d, J 7.9 Hz), 7.35 (1H, s), 7.23-7.11 (2H, m), 4.17 (1H, t, J6.3 Hz), 3.49 (3H, s), 3.20 (3H, s) and 3.13-3.11 (2H, m).

### Intermediate 4

(2S)-2-[(2-Chloropyridine-3-carbonyl)-amino]-3-(1H-indol-3-yl)-propionic acid methyl ester

A solution of 2-chloronicotinoyl chloride (1.0 g, 5.7 mmol) in dioxane (10 ml) was added slowly to a mixture of L-tryptophan methyl ester hydrochloride (1.59 g, 6.23 mmol), NaHCO₃ (1.43 g, 17.0 mmol), water (20 ml) and dioxane (10 ml). After 4 h at RT more NaHCO₃ (400 mg) and 2-chloronicotinoyl chloride (300 mg) were added. After a further 30 min the dioxane was removed in vacuo and the aqueous residue extracted with DCM. The extract was dried (MgSO₄) and concentrated in vacuo to give the title compound as a white solid (2.5g). δ_{H} (CDCl₃) 8.43 (1 H, dd, J 4.8, 2.0 Hz), 8.11 (1 H, s), 8.00 (1H, dd, J7.7, 2.0 Hz), 7.57 (1H, d, J7.8 Hz), 7.35 (1H, d, J8.1 Hz), 7.29 (1H, dd, J7.7, 4.8 Hz), 7.19 (1H, t, J7.0 Hz), 7.12-7.07 (3H, m), 5.12 (1H, m), 3.75 (3H, s), 3.52 (1H, dd, J 5.8, 14.5 Hz) and 3.43 (1H, dd, J 5.5, 15.0 Hz); m/z (ES⁺) 358.1 (MH⁺).

### Intermediate 5

(2S)-(1,3-Dioxo-1 ,3-dihydro-isoindol-2-yl)-3-(1H-indol-3-yl)-propionic acid methyl ester

Et₃N (2.2 ml, 15.7 mmol) and phthalic anhydride (1.3 g, 8.6 mmol) were added to tryptophan methyl ester hydrochloride (2.0 g, 7.9 mmol) in toluene (25 ml).

The mixture was heated at reflux for 3 days. The solvent was removed in vacuo. Purification by column chromatography (SiO₂, DCM) gave the title compound as a yellow solid (2.52 g, 92%). δ_{H} (CDCl₃) 8.02 (1H, br s), 7.78-7.72 (2H, m), 7.69-0.763 (2H, m), 7.60 (1 H, d, J 7.7 Hz), 7.28-7.25 (1 H, m), 7.15-7.00 (3H, m), 5.30-5.25 (1H, m), 3.80 (3H, s), 3.77-3.74 (2H, m).

### Intermediate 6

(2S)-(1,3-Dioxo-1 ,3-dihydro-isoindol-2-yl)-3-(1-ethanesulfonyl-1H-indol-3-yl)-propionic acid methyl ester

n-Butyl lithium (2.5 M in hexanes, 460 μl, 1.15 mmol) was added to a solution of Intermediate 5 (200 mg, 0.57 mmol) in THF (2 ml) at −78° C. After 15 min, ethanesulfonyl chloride (109 μl, 1.15 mmol) was added and the mixture allowed to warm slowly to RT overnight. The mixture was diluted with ether (20 ml) and washed with sat. NaHCO₃ and brine. The organic phase was dried (MgSO₄) and concentrated in vacuo. Purification by column chromatography (SiO₂, DCM) gave the title compound as a white solid (127 mg, 50%). m/z (ES⁺) 441.1 (MH⁺).

### Intermediate 7

(2S)-2-Amino-3-(1-ethanesulfonyl-1H-indol-3-yl)-propionic acid methyl ester

A mixture of Intermediate 6 (117 mg, 0.27 mmol) and hydrazine monohydrate (14 μl, 0.29 mmol) in abs. EtOH (4 ml) was stirred for 2 h at RT then heated at reflux overnight. The mixture was cooled to 0° C. then filtered. The filtrate was concentrated in vacuo, purification by column chromatography (SiO₂, 1% to 2% MeOH in DCM) gave the title compound, as a mixture with the corresponding ethyl ester, as a colourless oil (68 mg, 82%). δ_{H} (CDCl₃) 7.89-7.86 (1H, m), 7.63-7.59 (1H, m), 7.37-7.26 (3H, m), 4.18-4.08 (1H, m), 3.84-3.77 (m), 3.68 (s), 3.26 (2H, q, J 7.4 Hz), 3.22-3.15 (1H, m), 3.00 (1H, dd, J 7.4, 14.5 Hz), 1.21 (t, J 7.2 Hz), 1.18 (3H, t, J 7.4 Hz); m/z (ES⁺⁾ 311.1 (MH⁺ Me ester) 325.1 (MH⁺ Et ester).

### Intermediate 8

1 -Methanesulfonyl-1H-indole-3-carbaldehyde

1H-indole-3-carbaldehyde (14.5 g, 100 mmol) in DMF (100 ml) was added to a suspension of sodium hydride (60% in oil, 4.4 g, 110 mmol) in DMF (150 ml) at 0° C. After 1 h, methane sulfonyl chloride (8.51 ml, 11 Ommol) was added slowly. The mixture was stirred at 0° C. for 2 h then at RT for 2 h. Water was added to quench and the solvent removed in vacuo. The residue was dissolved in DCM, washed with water (x 2), dried (Na₂SO₄) and coricentrated in vacuo. Recrystallisation from EtOAc gave the title compound as brown crystals (9.2 g). δ_{H} (d₆-DMSO) 10.09 (1H, s), 8.61 (1H,s), 8.20-8.17 (1H, m), 7.92-7.89 (1 H, m), 7.53-7.42 (2H, m), 3.67 (3H, m); m/z (ES⁺) 224 (MH⁺).

### Intermediate 9

2-tert-Butoxycarbonylam i no-3-(1 -methanesulfonyl-1H-indol-3-yl)-acrylic acid methyl ester

A mixture of Intermediate 8 (7.0 g, 31.4 mmol), tert-Butoxycarbonylamino-(diethoxy-phosphoryl)-acetic acid methyl ester (10.2 g, 31.4 mmol) and DBU (4.69 ml, 31.4 mmol) in DCM (150 ml) was stirred at RT for 24 h. The mixture was diluted with DCM (200 ml), washed 1 M HCl, dried (Na₂SO₄) and concentrated in vacuo. Recrystallisation from EtOAc gave the title compound as pale yellow needles (7.65 g, 62%). δ_{H} (d₆-DMSO) 8.76 (1H, br s), 8.00 (1H, s), 7.88-7.85 (2H, m), 7.48-7.35 (3H, m), 3.76 (3H, s), 3.49 (3H, s), 1.40 (9H, s); m/z(ES⁺) 417 (MH⁺).

### Intermediate 10

2-tert-Butoxycarbonylamino-3-(1 -methanesulfonyl-1H-indol-3-yl)-propionic acid methyl ester

Wilkinson's catalyst (117 mg, 1 mol%) was added to a suspension of Intermediate 9 (5.0 g, 12.7 mmol) in MeOH (200 ml). The mixture was hydrogenation in a Parr apparatus at 50 p.s.i. at 50° C. for 24 h. More catalyst (117 mg) was added and hydrogenation continued as before for 24 h. The solvent was removed in vacuo. Purification by column chromatography (SiO₂, EtOAc) gave the title compound as a brown solid (4.62 g, 92%). δ_{H} (d₆-DMSO, 400 MHz) 7.81 (1H, d, J 8.1 Hz), 7.65 (1H, d, J 7.4 Hz), 7.45-7.31 (4H, m), 4.33-4.27 (1H, m), 3.63 (3H, s), 3.31 (3H, s), 3.14 (1H, dd, J4.7, 14.7 Hz), 3.02 (1H, dd, J9.9, 14.7 Hz), 1.32 (9H, s); m/z (ES⁺) 419.2 (Mna⁺).

### Intermediate 11

2-Amino-3-(1-methanesulfonyl-1H-indol-3-yl)-propionic acid methyl ester hydrochloride

Gaseous HCl was bubbled through a solution of Intermediate 10 (4.62 g, 11.7 mmol) in EtOAc (100 ml) for a few seconds. The mixture was stirred at RT for 30 min. The mixture was re-treated with HCl until reaction was complete as judged by t.l.c. The solid was filtered off and dried to give the title compound as a white solid (3.41 g, 88%). 5H (d₆-DMSO, 400 MHz) 8.72 (3H, br s), 7.87 (1H, d, J 8.2 Hz), 7.69 (1H, d, J 7.7 Hz), 7.59 (1H, s), 7.46-7.36 (2H, m), 4.41 (1H, t, J 6.4 Hz), 3.74 (3H, s), 3.43 (3H, s), 3.3 (2H, obscured by HOD signal); m/z (ES⁺) 297.0 (MH⁺).

### Intermediate 12

2-Chloro-terephthalic acid 4-methyl ester

A solution of boron tribromide (1 M in DCM, 38.9 ml, 38.9 mmol) was added slowly to dimethyl chlorophthalate (8.9 g, 38.9 mmol) in DCM (200 ml) at −50° C. The mixture was allowed to warm to RT overnight then poured onto ice and partitioned with EtOAc. The organic phase was concentrated in vacuo, the residue dissolved in aq NaHCO₃ and extracted with DCM. The aqueous phase was acidified with c.HCl to pH 1 and the solid filtered off and dried to give the title compound as a white solid (contaminated with corresponding diacid) (8.4 g). δ_{H} (d₆-DMSO) 8.05-7.84 (3H, m), 3.88 (3H, s); m/z(ES⁺) 214.9 (MH⁺).

### Intermediate 13

2-Chloro-terephthalic acid 1-tert-butyl ester 4-methyl ester

N,N-Dimethylformamide di-tert-butyl acetal (38 ml, 160 mmol) was added slowly at reflux to a suspension of Intermediate 12 (8.24 g, 38 mmol) in toluene (100 ml). The mixture was heated at reflux for a further 3 h then diluted with EtOAc, washed with aq. NaHCO₃, water and brine, dried (Na₂SO₄) and concentrated in vacuo. Purification by column chromatography (SiO₂, 7% EtOAc in hexane) gave the title compound as a colourless oil (6.13 g, 60%). δ_{H}(CDCl3) 8.07 (1H, d, J 1.6 Hz), 7.93 (1H, dd, J 1.6, 8.1 Hz), 7.74 (1 H, d, J 8.1 Hz), 3.94 (3H, s), 1.61 (9H, s); m/z (ES⁺) 293 (Mna⁺).

### Intermediate 14

2-Chloro-terephthalic acid 1-tert-butyl ester

Lithium hydroxide monohydrate (1.40 g, 33.3 mmol) was added to a solution of Intermediate 13 (6.0 g, 22.2 mmol) in THF/water (3:1, 100 ml). After 90 min at RT the THF was removed in vacuo. The aqueous residue was diluted with water and extracted with ether. The aqueous phase was acidified with c.HCl and extracted with DCM. The organic extracts were dried (Na₂SO₄) and concentrated in vacuo to give the title compound as a white solid (5.44 g, 96%). δ_{H} (d₆-DMSO) 13.55 (1H, br s), 7.92-7.60 (2H, m), 7.79 (1H, d, J 7.9 Hz), 1.55 (9H, s); m/z (ES⁺) 279 (Mna⁺).

### s Intermediate 15

2-Chloro-N-(3-methoxy-benzyl)-terephthalamic acid tert-butyl ester

EDC was added to a mixture of Intermediate 14 (5.44 g, 21 mmol), 3-methoxybenzylamine (2.88 g, 21 mmol), HOBT (3.12 g, 23.1 mmol) and NMM (2.54 g, 23.1 mmol) in DCM (100 ml). The mixture was stirred overnight at RT then diluted with DCM, washed with 2M HCl, aq. NaHCO₃ and water, dried (Na₂SO₄) and concentrated in vacuo. Recrystallisation from EtOAc gave the title compound as white needles (5.99 g, 76%). δ_{H} (d₆-DMSO) 9.22 (1 H, br t, J 5.8 Hz), 8.00 (1H, d, J 1.6 Hz), 7.90 (1H, dd, J 1.6, 8.1 Hz), 7.78 (1H, d, J 8.0 Hz), 7.23 (1H, t, J7.3 Hz), 6.89-6.87 (2H, m), 6.82-6.79 (1H, m), 4.44 (2H, d, J5.7 Hz), 3.72 (3H, s), 1.54 (9H, s); m/z(ES⁺) 376 (MH⁺).

### Intermediate 16

2-Chloro-N-(3-hydroxy-benzyl)-terephthalamic acid

Boron tribromide (1 M in DCM, 3.99 ml, 3.99 mmol) was added dropwise to a solution of Intermediate 15 (500 mg, 1.33 mmol) in DCM at −5° C. The mixture was stirred at RT overnight then partitioned between EtOAc and water. The organic phase was dried (Na₂SO₄) and concentrated in vacuo to give the title compound as a yellow oil (509 mg). δ_{H} (d₆-DMSO) 9.31 (1H, br s), 9.21 (1H, t, J 5.9 Hz), 8.01 (1H, d, J 1.4 Hz), 7.92-7.84(2H, m), 7.10 (1H, t, J 8.1 Hz), 6.74-6.71 (2H, m), 6.64-6.60 (1H, m), 4.39 (2H, d, J 5.9 Hz); m/z (ES⁺) 306 (MH⁺).

### Intermediate 17

(1-Methanesulfonyl-1H-indol-3-yl)-methanol

Sodium borohydride (851 mg, 22.4 mmol) was added in portions to a suspension of Intermediate 8 (2.50 g, 11.2 mmol) in abs. EtOH (25 ml) at 0° C. After 2 h at RT, the solvent was removed in vacuo. Aq. NaOH (1 M) was added to the residue and the mixture extracted with ether (x 2). The ether extracts were dried (Na₂SO₄) and concentrated in vacuo to give the title compound as a white solid (2.49 g, quant). δ_{H} (d₆-DMSO) 7.81 (1H, d, J 8.2 Hz), 7.71 (1H, d, J7.1 Hz), 7.44 (1H, s), 7.40-7.27 (2H, m), 5.13 (1H, br S), 4.65 (2H, s), 3.36 (3H, s); m/z (ES⁺) 207.9 (M⁺-(H₂O )).

### Intermediate 18

3-Bromomethyl-1-methanesulfonyl-1H-indole

Anhydrous HBr was bubbled through a suspension of Intermediate 17 (2.45 g, 10.9 mmol) in ether (100 ml) for a few minutes. The mixture was then stirred at RT for 20 min. The solvent was removed in vacuo to give the title compound as a pink solid. δ_{H} (d₆-DMSO) 7.95-7.75 (3H, m), 7.48-7.35 (2H, m), 4.95 (2H, s), 3.47 (3H, s); m/z (ES⁺) 208 (M⁺-Br.

### Intermediate 19

2-Amino-3-(1 -methanesulfonyl-1H-indol-3-yl)-propionic acid ethyl ester

A solution of LDA (2 M, 6.0 ml, 12 mmol) was added to a solution of N-(diphenylmethylene)glycine ethyl ester (2.91 g, 10.9 mmol), in THF (70 ml) at −78° C. After 1 h a solution of Intermediate 18 (3.14 g, 10.9 mmol) in THF (30 ml) was added. The mixture was stirred for 2 h at −78° C. then allowed to warm to RT over 2 h. Water was added, the bulk of the THF removed in vacuo and the residue partitioned between EtOAc and water. The organic phase was washed with brine, dried (Na₂SO₄) and concentrated in vacuo to give a light brown gum. This was dissolved in THF (100 ml) and treated with 1 M HCl (100 ml). After 30 min the THF was removed in vacuo. The aqueous residue was extracted with ether (3×100 ml), basified with NaOH (pH10) and extracted with DCM. The DCM extracts were dried (Na₂SO₄) and concentrated in vacuo to give the title compound as a yellow oil (2.03 g). δ_{H} (d₆-DMSO) 7.80 (1H, d, J 7.5 Hz), 7.64 (1H, d, J 7.0Hz), 7.38 (1H, s), 7.37-7.27 (2H, m), 4.00 (2H, q, J 7.1 Hz), 3.65 (1 H, t, J 6.6 Hz), 3.32 (3H, s), 3.00 (1H, dd, J 6.1, 14.4 Hz), 2.90 (1H, dd, J 7.1, 14.4 Hz), 1.84 (2H, br s), 1.08 (3H, t, J 7.2 Hz); m/z (ES⁺) 311 (MH⁺).

### EXAMPLE 1

(2S)-2-[(3,5-Dichloropyridine-4-carbonyl)-aminol-3-(1-methanesulfonyl-1H-indol-3-yl)-propionic acid methyl ester

A solution of 3,5-dichloroisonicotinoyl chloride (168 mg, 0.8 mmol), Intermediate 3 (250 mg, 0.75 mmol) and Et₃N (111μl, 0.8 mmol) in DCM (15 ml) was stirred a RT for 5 h. The mixture was diluted with DCM (50 ml), washed with sat. NaHCO₃ (50 ml), dried (Na₂SO₄) and concentrated in vacuo to give the title compound as a white foam (376 mg, 100%). δ_{H} (CDCl₃) 8.95 (2H, s), 7.89 (1H, d, J 8.3 Hz), 7.66 (1H, d, J 8.3 Hz), 7.43-7.28 (3H, m), 6.74 (1H, br d, J 7.5 Hz), 5.27-5.20 (1H, m), 3.77 (3H, s), 3.44-3.38 (2H, m) and 3.08 (3H, s).

### Example 2

(2S)-2-(2,6-Dichlorobenzoylamino)-3-(1 -methanesulfonyl-1H-indol-3-yl)-propionic acid methyl ester

A solution of 2,6-dichlorobenzoyl chloride (160 mg, 0.75 mmol), Intermediate 3 (250 mg, 0.75 mmol) and Et₃N (210 μl, 1.5 mmol) in DCM (15 ml) was stirred a RT for 2 h. The mixture was diluted with DCM (60 ml), washed with 2 M HCl (50 ml), dried (Na₂SO₄) and concentrated in vacuo to give the title compound as a white foam (375 mg, 100%). δ_{H} (CDCl₃) 7.90 (1H, d, J 7.9 Hz), 7.69 (1H, d, J 8.4 Hz), 7.41-7.27 (6H, m), 6.48 (1H, br d, J 7.6 Hz), 5.27-5.23 (1H, m), 3.73 (3H, s), 3.42 (2H, d, J 5.7 Hz) and 3.07 (3H, s).

### Example 3

(2S)-2-[(2-Chloropyridine-3-carbonyl)-amino]-3-(1-methanesulfonyl-1H-indol-3-yl)-propionic acid methyl ester

Methanesulfonyl chloride (195 μl, 2.5 mmol) was added to a solution of Intermediate 4 (750 mg, 2.1 mmol) and Et₃N (350 μl, 2.5 mmol) in DCM (11 ml) and the mixture stirred at RT. A further 2 equivalents of methane sulfonyl chloride and Et₃N were added in portions during the course of the 16 h reaction. The mixture was diluted with DCM (10 ml), washed with sat. NaHCO₃ and water, dried (MgSO₄) and concentrated in vacuo. Purification by column chromatography (SiO₂; 1.5% MeOH in DCM) gave the title compound as a yellow solid (144 mg). δ_{H} (CDCl₃) 8.47 (1H, dd, J4.7, 2.0 Hz), 8.04 (1H, dd, J 7.7, 2.0 Hz), 7.90 (1H, d, J 8.3 Hz), 7.61 (1H, d, J 7.7 Hz), 7.38-7.14 (5H, m), 5.16-5.14 (1H, m), 3.77 (3H, s), 3.47 (1H, dd, J 14.6, 5.7 Hz), 3.37 (1H, dd, J14.6, 5.7 Hz) and 3.05 (3H, s); m/z(ES⁺) 436.1 (MH⁺).

### Example 4

(2S)-2-[(3,5-Dichloropyridine-4-carbonyl)-amino]-3-(1-methanesulfonyl-1H-indol-3-yl)-propionic acid

Lithium hydroxide monohydrate (33.6 mg, 0.8 mmol) was added to the compound of Example 1 (356 mg, 0.75 mmol) in a mixture of THF (10 ml) and water (10 ml). After 6 h at RT the mixture was concentrated in vacuo. The residue was diluted with water and acidified with 2 M HCl (1 ml). The precipitate was filtered off, washed with water and dried to give the title compound as a white solid (168 mg, 49%). δ_{H} (d₆-DMSO) 12.95 (1H, br s), 9.32 (1H, d, J 8.0 Hz), 8.65 (2H, s), 7.82 (1H, d, J 8.0 Hz), 7.72 (1H, d, J 7.3 Hz), 7.45 (1H, s), 7.42-7.33 (2H, m), 4.87-4.81 (1H, m), 3.31 (3H, s), 3.31-3.28 (1H, m) and 3.16-3.10 (1H, m); m/z(ES⁺) 456 (MH⁺).

### Example 5

(2S)-2-(2.6-Dichlorobenzoylamino)-3-(1 -methanesulfonyl-1 H-indol-3-yl)-propionic acid

From the compound of Example 2 by the method of Example 4. White solid (243 mg, 67%). δ_{H} (d₆-DMSO) 12.80 (1H, br s), 9.12 (1H, d, J 8.0 Hz), 7.81 (1 H, d, J 7.9 Hz), 7.71 (1 H, d, J 7.9 Hz), 7.45-7.32 (6H, m), 4.82-4.77 (1H, m), 3.31 (3H, s), 3.29-3.24 (1H, m), 3.10 (1H, dd, J9.6, 15.3 Hz); m/z (ES⁺) 455.0 (Mna⁺).

### Example 6

(2S)-2-[(2-Chloropyridine-3-carbonyl)-amino]-3-(1-methanesulfonyl-1 H-indol-3-yl)-propionic acid

From the compound of Example 3 by the method of Example 4. Off-white solid (108 mg, 77%). δ_{H} (d₆-DMSO) 12.90 (1H, br s), 8.87 (1H, d, J 8.0 Hz), 8.26 (1H, dd, J 4.8, 2.0 Hz), 7.63 (1 H, d, J 7.5 Hz), 7.54-7.47 (2H, m), 7.30-7.12 (4H, m), 4.59-4.52 (1H, m), 3.09-2.89 (2H, m) and 3.13 (3H, s); m/z (ES⁺) 422.0 (MH⁺).

### Example 7

(2S)-2-[(2,4-Dichloropyridine-3-carbonyl)-amino]-3-(1 -methanesulfonyl-1 H-indol-3-yl)-propionic acid methyl ester

A mixture of Intermediate 3 (149 mg, 0.45 mmol), HOBT (77 mg, 0.57 mmol), NMM (125 μl, 1.14 mmol), 2,4-dichloronicotinic acid (78 mg, 0.41 mmol) and EDC (109 mg, 0.57 mmol) in DCM (4.5 ml) was stirred at RT for 5h. The mixture was diluted with DCM and washed with 2M HCl, sat. NaHCO₃ and water. The organic phase was dried (MgSO₄) and concentrated in vacuo. Purification by column chromatography (SiO₂, 1% to 5% MeOH in DCM) gave the title compound as a white solid (187 mg, 88%). 8H (CDCl3) 8.04 (1 H, d, J8.1 Hz), 7.92 (1H, d, J8.2 Hz), 7.61 (1H, d, J9.0 Hz), 7.43-7.28 (4H, m), 7.18 (1H, br d), 5.15 (1H, dt, J7.1, 5.7 Hz), 3.79 (3H, s), 3.51 (1H, dd, J5.7, 14.8 Hz), 3.37 (1H, dd, J5.7, 14.8 Hz), 3.08 (3H, s); m/z(ES⁺) 469.9 (MH⁺).

### Example 8

(2S)-2-[(2,4-Dichloropyridine-3-carbonyl)-amino]-3-(1 -methanesulfonyl-1 H-indol-3-yl)-propionic acid

From the compound of Example 7 by the method of Example 4. δ_{H} (d₆-DMSO) 9.06 (1 H, d, J 7.9 Hz), 7.80 (1H, d, J 7.9 Hz), 7.74-7.69 (2H, m), 7.61 (1 H, d, J 8.0 Hz), 7.43 (1 H, s), 7.40-7.29 (2H, m), 4.72 (1 H, m), 3.3 (3H, s), 3.3 (1H, signal obscured), 3.11 (1H, dd, J 9.4, 15.1 Hz); m/z (ES⁺) 455.9 (MH⁺).

### Example 9

(2S)-2-[(2-Chloropyridine-3-carbonyl)-amino]-3-(1 -ethanesulfonyl-1 H-indol-3-yl)-propionic acid methyl ester

2-Chloronicotinoyl chloride (39 mg, 0.22 mmol) was added to a solution of Intermediate 7 (68 mg, 0.21 mmol) and Et₃N (31μl, 0.22 mmol) in THF (4 ml). After 15 min the solvent was removed in vacuo, the residue partitioned between EtOAc and water and the aqueous phase re-extracted with EtOAc. The combined organics were washed with water, dried (MgSO₄) and concentrated in vacuo. Purification by column chromatography (SiO₂, 2% MeOH in DCM) gave the title compound, as a mixture with the corresponding ethyl ester, as a colourless oil. δ_{H} (CDCl3) 8.40 (1 H, dd, J 2.0, 4.8 Hz), 7.97 (1H, dd, J2.0, 7.6 Hz), 7.86-7.83 (1H, m), 7.61-7.57 (1H, m), 7.35-7.23 (5H, m), 5.14-5.06 (1H, m), 4.23-4.12 (m), 3.73 (s), 3.46 (1H, dd, J 5.9, 14.8 Hz), 3.34 (1H, dd, J5.8, 14.8 Hz), 3.25 (2H, q, J7.4 Hz), 1.23 (3H, t, J7.2 Hz), 1.14 (t, J 6.8 Hz).

### Example 10

(2S)-2-[(2-Chloropyridine-3-carbonyl)-amino]-3-(1 -ethanesulfonyl-1 H-indol-3-yl)-propionic acid

From the compound of Example 9 by the method of Example 4. Light pink solid. δ_{H} (d₆-DMSO, 400 MHz, 350K) 8.70 (1H, d, J 8.0 Hz), 8.44 (1H, dd, J 1.9, 4.8 Hz), 7.86-7.84 (1 H, m), 7.74-7.69 (2H, m), 7.47-7.40 (2H, m), 7.38-7.31 (2H, m), 4.80 (1 H, dt, J 5.3, 8.4 Hz), 3.47 (2H, q, J 7.3 Hz), 3.34 (1H, dd, J4.4, 15.1 Hz), 3.19 (1H, dd, J8.9, 15.1 Hz), 1.08 (3H, t, J7.3 Hz); m/z (ES⁺) 436.1 (MH⁺).

### Example 11

2-[2-Chloro-4-(3-hydroxy-benzylcarbamoyl)-benzoylamino]-3-(1 -methanesulfonyl-1 H-indol-3-yl)-propionic acid methyl ester

EDC (106 mg, 0.55 mmol) was added to a mixture of Intermediate 16 (153 mg, 0.5 mmol), Intermediate 11 (166 mg, 0.5 mmol), HOBT (74 mg, 0.55mmol) and NMM (115 μl, 1.05 mmol) in DMF (5ml). The mixture was stirred at RT overnight. The solvent was removed in vacuo. The residue was dissolved in EtOAc and washed with 2M HCl, aq. NaHCO₃ and brine, dried (Na₂SO₄) and concentrated in vacuo. Trituration with 5% MeOH in DCM gave a solid which was filtered off and dried to give the title compound as a white solid (207 mg, 71%). δ_{H} (d₆-DMSO) 9.30 (1H, s), 9.15 (1H, br t), 9.07 (1H, d, J7.8 Hz), 7.95 (1 H, d, J 1.6 Hz), 7.86-7.81 (2H, m), 7.70 (1 H, d, J 7.0 Hz), 7.47 (1 H, s), 7.42-7.32 (3H, m), 7.10 (1H, t, J8.0 Hz), 6.72-6.70 (2H, m), 6.62 (1H, d, J9.2 Hz), 4.82-4.78 (1 H, m), 4.38 (2H, br), 3.69 (3H, s), 3.32 (3H, s), 3.3 (1 H, obscured by HOD), 3.16 (1H, dd, J10.8,14.2 Hz); m/z(ES⁺) 584.0 (MH⁺).

### Example 12

2-[2-Chloro-4-(3-hydroxy-benzylcarbamoyl)-benzoylamino]-3-(1 -methanesulfonyl-1 H-indol-3-yl)-propionic acid

From the compound of Example 11 by the method of Example 4. White solid. δ_{H} (d₆-DMSO, 400 MHz) 13.13 (1H, br s), 9.46 (1H, br s), 9.30 (1 H, t, J 5.9 Hz), 9.08 (1 H, d, J 8.0 Hz), 8.09 (1 H, d, J 1.4Hz), 8.01-7.96 (2H, m), 7.87 (1H, d, J 7.7 Hz), 7.61 (1H, s), 7.56-7.47 (3H, m), 7.25 (1H, t, J 7.9 Hz), 6.87-6.85 (2H, m), 6.78-6.76 (1 H, m), 4.91-4.86 (1 H, m), 4.53 (2H, d, J 5.8 Hz), 3.45 (3H, s), 3.4 (1H, obscured by HOD), 3.28 (1H, dd, J 10.0, 15.0 Hz); m/z(ES⁺) 570.0 (MH⁺).

Solution phase parallel synthesis general method

Reaction of a variety of (hetero)aryl acid chloride (prepared from corresponding acid if necessary) with the amino ester Intermediate 19, followed by ester hydrolysis and purification to give the acids. The (hetero)aryl acid (0.15 mmol) in DCM was treated with oxalyl chloride (26μl, 0.3mmol) for 3-4h at RT. The solvent and excess reagent were removed in vacuo to give the acid chloride. A solution of Intermediate 19 (31 mg, 0.1 mmol) in THF (0.5 ml) was added to a mixture of the acid chloride (0.3 mmol) and DIEA (35 μl, 0.2 mmol) in THF (1.5 ml). After 2.5h at RT, PS-Trisamine (80 mg, 0.3 mmol) and MP-TsOH (280 mg, 0.4 mmol) were added. After 3h at RT the resins were filtered off and washed with THF (4×0.5 ml). Lithium hydroxide monohydrate (4.2 mg, 0.1 mmol) in water (0.5ml) was added to the filtrate. After 2h at RT, glacial 5 acetic acid (12 μl, 0.2 mmol) was added then the mixture concentrated in vacuo. Purification by HPLC gave the series of acids listed below.

Analytical HPLC method used for characterisation

| Equipment: | Hewlett Packard 1100 LC/MSD | | |
|---|---|---|---|
| Method: | Mobile Phase | A:-0.1% formic acid | |
| | | B:-0.1% formic acid in acetonitrile | |
| | Gradient:- | Time(min) | % B |
| | | Initial | 5 |
| | | 3.0 | 95 |
| | | 5.0 | 95 |
| | | 5.5 | 5 |

| | |
|---|---|
| Stop | 7.00 |

| | Flow Rate: | 0.9 ml/min |
|---|---|---|
| | Column Temp: | 40° C. |
| | Column: | Phenomenex Luna 3 μ C₁₈(2) |
| | | 50 × 4.6 mm |
| Detection | UV DAD | 210-450 nm step 2 nm |
| | MS | ES + ve |
| | Mode scan | 120-1000 |
| | Peak width | 0.1 min⁻¹ |
| | Fragmentor | 80 V |
| | Drying gas | 13.0 l/min |
| | flow rate | |
| | Nebuliser | 50 psig |
| | Pressure | |
| | Drying gas | 350° C. |
| | temperature | |
| Software | Chemstation | Vs 8.03 |
| | Software | |

### Example 13

3-(l -Methanesulfonyl-1 H-indol-3-yl)-2-[(2-methoxy-pyridine-3-carbonyl)-amino]-propionic acid

From 2-methoxy-nicotinic acid. Retention time 3.235 min; m/z (ES⁺) 418.0 (MH⁺).

### Example 14

2-(2-Chloro-benzoylamino)-3-(1 -methanesulfonyl-1 H-indol-3-yl)-propionic acid

From 2-chloro-benzoic acid. Retention time 3.307 min; m/z (ES⁺) 421.0 (MH⁺).

### Example 15

3-(1 -Methanesulfonyl-1 H-indol-3-yl)-2-(2-trifluoromethyl-benzoylamino)-propionic acid

From 2-trifluoromethyl-benzoic acid. Retention time 3.396 min; m/z (ES⁺) 455.0 (MH⁺).

### Example 16

2-(2,6-Dimethoxy-benzoylamino)-3-(1 -methanesulfonyl-1 H-indol-3-yl)-propionic acid

From 2,6-dimethoxy-benzoic acid. Retention time 3.188 min; m/z(ES⁺) 447.0(MH⁺).

### Example 17

2-[(2-Chloro-6-methyl-pyridine-3-carbonyl)-amino]-3-(1 -methanesulfonyl-1 H-indol-3-yl)-propionic acid

From 2-chloro-6-methyl-nicotinic acid. Retention time 3.115 min; m/z (ES⁺) 436.0 (MH⁺).

### Example 18

2-(2-Bromo-5-nitro-benzoylamino)-3-(1 -methanesulfonyl-1 H-indol-3-yl)-propionic acid

From 2-bromo-5-nitro-benzoic acid. Retention time 3.421 min; m/z (ES⁺) 511.9 (MH⁺).

### Example 19

2-[(2-Ethylsulfanyl-pyridine-3-carbonyl)-amino]-3-(1 -methanesulfonyl-1H-indol-3-yl)-propionic acid

From 2-ethylsulfanyl-nicotinic acid. Retention time 3.365 min; m/z (ES⁺) 448.0 (MH⁺).

### Example 20

2-(2,6-Dichloro-benzoylamino)-3-(1 -methanesulfonyl-1 H-indol-3-yl)-propionic acid

From 2,6-dichloro-benzoyl chloride. Retention time 3.359 min; m/z (ES⁺) 455.0 (MH⁺).

### Example 21

2-[(3,5-Dichloro-pyridine-4-carbonyl)-amino]-3-(1 -methanesulfonyl-1 H-indol-3-yl)-propionic acid

From 3,5-dichloro-isonicotinoyl chloride. Retention time 3.191 min; m/z (ES⁺) 456.0 (MH⁺).

### Example 22

2-[(2-Chloro-pyridine-3-carbonyl)-amino]-3-(1 -methanesulfonyl-1 H-indol-3-yl)-propionic acid

From 2-Chloro-nicotinoyl chloride. Retention time 3.037 min; m/z (ES⁺) 422.0 (MH⁺)

The following assays can be used to demonstrate the potency of the compounds according to the invention. In each of these assays an lC₅₀ value was determined for each test compound and represents the concentration of compound necessary to achieve 50% inhibition of adhesion where 100% =adhesion assessed in the absence of test compound and 0% =adhesion in the absence of cells or ICAM-1.

LFA-1 -Dependent Cell Assay

96 well Nunc immunoplates were coated overnight with affinipure F(ab)₂ goat anti-human lgG Fc (Jackson lmmunoResearch) at 2μg/ml in Dulbecco's phosphate-buffered saline (PBS). Plates were then blocked for lh at RT with 1% (w/v) bovine serum albumin (BSA) in PBS, and incubated with 200 ng/ml of 5-domain lCAM-1-human Fc construct in PBS for 2h. Inhibitors were serially diluted across plates in assay medium (RPMl 1640+10% foetal calf serum), 3×10⁵ HL60 cells added together with phorbol-12-myristate-13-aceteate (PMA) at 20 ng/ml in a total volume of 200μl. After 30 minutes incubation at 37° C., plates were washed twice in assay medium, adherent cells fixed in methanol and stained with 0.25% (w/v) Rose Bengal in PBS. s After removal of unbound dye, bound dye was liberated with 100μl 1:1 PBS:ethanol and absorbance read at 570nm.

LFA-1 -Dependent Protein-Protein Assay

96 well Nunc immunoplates were coated overnight with an anti-β2 integrin monoclonal antibody (KIM185) at 5 μg/ml in PBS. After blocking plates for 1 h in PBS/2% BSA/1% Tween 20, 100μl of a lysate from HL60 cells in 20 mM Tris/150 mM NaCl/1mM MnCl_{2/1}% Nonidet P-40 was added for 3h at RT. After washing, wells received lCAM-1 -human Fc (final concentration 2μg/ml) in the presence of serial dilutions of inhibitors in conjugate buffer (20mM Tris/150mM NaCl/ 1 mM MnCl₂/1 % (w/v) ovalbumin) and plates were incubated for 2h at RT. After further washes, plates were incubated with a peroxidase-conjugated F(ab)₂ goat anti-human lgG Fc (Jackson ImmunoResearch) in conjugate buffer for 1 hour, washed finally, and the signal developed using TM Blue substrate with absorbance read at 630 nm.

In the above cell assay compounds of the invention generally have lC₅₀ values of 100μM and below. In the above protein assay compounds of the invention generally have lC₅₀ values of 1 μM and below.

Some of the preferred embodiments of the invention described above are outlined below and include, but are not limited to, the following embodiments. As those skilled in the art will appreciate, numerous changes and modifications may be made to the preferred embodiments of the invention without departing from the spirit of the invention. It is intended that all such variations fall within the scope of the invention.

The entire disclosure of each patent and publication cited herein is hereby incorporated by reference.

1. A compound of formula (1): 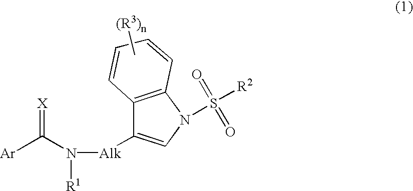
wherein:
Ar is an optionally substituted aromatic or heteroaromatic group;
X is an oxygen or sulphur atom;
Alk is a chain 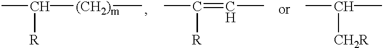
in which m is zero or the integer 1or 2 and R is a carboxylic acid (—CO₂H) or a derivative or biostere thereof;
R¹ is a hydrogen atom or a C₁₋₆alkyl group;
R² is an optionally substituted aliphatic group;
R³ is an atom or group —L¹(Alk^{l})ₜL²(R⁴)^{u}, in which L¹ and L² which may be the same or different is each a covalent bond or a linker atom or group, t is zero or the integer 1, u is an integer 1, 2 or 3, Alk¹ is an aliphatic or heteroaliphatic chain and R⁴ is a hydrogen or halogen atom or a group selected from alkyl, —OR⁵ [where R⁵ is a hydrogen atom or an optionally substituted alkyl group], —SR⁵, —NR⁵R⁶ [where R⁶ is as just defined for R⁵ and may be the same or different], —NO₂, —CN, —CO₂R⁵, —SO₃H, —SOR⁵, —SO₂R⁵, —SO₃R⁵, —OCO₂R⁵, —CONR⁵R⁶, —OCONR⁵R⁶,—CSNR⁵R⁶, —COR⁵, —OCOR⁵, —N(R⁵)COR⁶, —N(R⁵)CSR⁶, —N(R⁵)CO₂R⁶, SO₂N(R ⁵)(R ⁶), —N(R⁵)SO₂R⁶, —N(R⁵)CON(R⁶)(R⁷) [where R⁷ is a hydrogen atom or an optionally substituted alkyl group], N(R⁵)CSN(R⁶)(R⁷) or —N(R⁵)SO₂N(R⁶)(R7), provided that when t is zero and each of L¹ and L² is a covalent bond then u is the integer 1 and R⁴ is other than a hydrogen atom;
n is zero or the integer 1, 2 or 3;
and the salts, solvates, hydrates and N-oxides thereof.

2. A compound according to claim 1 in which R¹ is a hydrogen atom.

3. A compound according to claim 1 in which Alk is a —CH(R)CH₂—group.

4. A compound according to claim 1 in which R is a carboxylic acid (—CO₂H) group.

5. A compound according to claim 1 in which R² is an optionally substituted C₁₋₆alkyl group.

6. A compound according to claim 1 in which Ar is an optionally substituted phenyl or pyridyl group.

7. A compound which is:
(2S)-2-[(3 ,5-dichloropyridine-4-carbonyl)-amino]-3-( 1 -methanesulfonyl-1 H-indol-3-yl)-propionic acid;
(2S)-2-(2,6-dichlorobenzoylamino)-3-(1 -methanesulfonyl-1 H-indol-3-yl)-propionic acid;
(2S)-2-[(2-chloropyridine-3-carbonyl)-amino]-3-(1 -methanesulfonyl- 1 H-indol-3-yl)-propionic acid;
2-[2-chloro-4-(3-hydroxy-benzylcarbamoyl)-benzoylamino]-3-(1 -methanesulfonyl- 1 H-indol-3-yl)-propionic acid;
2-(2,6-dichloro-benzoylamino)-3-(1 -methanesulfonyl-1 H-indol-3-yl)-propionic acid;
2-[(3 ,5-dichloro-pyridine-4-carbonyl)-amino]-3-( 1 -methanesulfonyl-1 H-indol-3-yl)-propionic acid;
and the salts, solvates, hydrates and N-oxides thereof.

8. A pharmaceutical composition comprising a compound according to claim 1 together with one or more pharmaceutically acceptable carriers, excipients or diluents.

9. A compound for the prophylaxis or treatment of a disease or disorder in a mammal in which inappropriate leukocyte trafficking plays a role, comprising administering to a mammal suffering from such a disease or disorder a therapeutically effective amount of a compound according to claim 1.

10. A method according to claim 9 wherein the disease or disorder is an acute or chronic inflammatory disease.

11. A method according to claim 9 wherein the disease or disorder is a inflammatory or hyperproliferative skin disease.

12. A method according to claim 11 wherein the inflammatory or hyperproliferative skin disease is selected from psoriasis, atopic dermatitis, allergic contact dermatitis, irritant contact dermatitis, eczematous dermatitis or seborrhoeic dermatitis.

13. A method for inhibiting, in a mammal, the binding of LFA-1 to the ligands thereof, comprising administering to the mammal an effective amount of a compound according to claim 1

14. A method according to claim 13 wherein the ligand is lCAM-1.

15. A method for the prophylaxis of treatment of a disease or disorder in a mammal in which inappropriate leukocyte trafficking plays a role, comprising administering to a mammal suffering from such a disease or disorder a therapeutically effective amount of compound according to claim 1.

16. A method according to claim 15 wherein the disease or disorder is an acute or chronic inflammatory disease.

17. A method according to claim 15 wherein the disease or disorder is n inflammatory or hyerproliferative skin disease.

18. A method according to claim 17 wherein the inflammatory or hyperproliferative skin disease is selected from psoriasis, atopic dermatitis, allergic contact dermatitis, irrtant contact dermnatitis, eczematous dermatitis or seborrhoeic dermatitis.
